SEQUENCE LISTING (18 sequences)

SEQ ID NO: 1 - protein, 947 aa, Arabidopsis thaliana [sequence omitted]

SEQ ID NO: 2 - DNA, 6726 bp, Arabidopsis thaliana
ctaacaactttggtgacagctaaagaaaaactaagttagaagaatatttagataaccttcaagctcgagaccactcggatctctctctaatcgccgatcggaatccgccatgggagtaccggcgttctacagatggctcgctgaccgatacccgaagtcaattagtgacgtcgttgaagaagaacctaccgatggaggtcgtggtgaccttattcctgttgatatcaccagacctaaccctaacggcttcgaatttgataatttgtatctcgatatgaatggtatcattcatccttgtttccatcctgaaggaaaggtatgttgtagctcagaaatgtttggttagggtttgagttgatttgttgtatgagaaatgaaatcaatgagtttagtagtttggtggtggagaagattattgttgtctgtattcgtagtttagtacagttatgagagaaggagaagtatactagttgttgcttttcctcgattttgcttcctgcacgtcaatggaagcgatggccaacttggtgatactagacgtagggtatctaacttttctgaagtgtatgattagagtttgagttgattgtattaggcagtaacaagtagacatggatcgtatgagaaatgaaatcactaagtttggtattttggtgagaagatgatttttttttggtgttcataatttagctcttttattggggaagggaggagtgtactagttgttgctttccgactatttttgcttaccgaaagtcaatagaagcaatggccatttttgtgatactagattttgggatctaaagttcagttcagaaagtgatttagatatattatttgggaaattggatgcttatggctgtgtgtattttgttcatgtatcacttatgcgagtctaaagtcagtttttttataaaaatagttgactagaaaattaaaagacactgatgttattagtttttcattatagttgaggagatattatcgaagggagtgagtttgcatagcaatacgtgttatggtgatacttgatttccattaaagattttactagctaatcgtactttgattacaatgcagcctgcacctgccacgtacgatgatgtatttaaatcaatgtttgagtacattgaccatctttttactttagtgcgtccgaggaagattctttatttggctataggtgatcttgtgtttcctttttatcatctgtctgttttgctctgtgagatgtggttgattaaatatactgttgcagatggagttgcgccgagagcaaagatgaatcagcagcgttctcgacgtttcagagctgcaaaggacgcagcagaggctgtatgttctctttgtgtttctatttttacatttactcttggctttacatgggtcactgttgcttatactatttctggtttaataggaagctgaggaagagaggttgagaaaagattttgagatggagggccaaatcttgtccgctaaagagaaagctgaaacctgtgattcaaatgtcattactcctgggaccccttttatggctatactgtcagtcgctcttcagtattacatccaatctaggttaaaccataaccctggctggagatatgttaaggtgtgatactgtttcatctaacttagcagctatttccaaactgtacaagacttttcgcattcttgcagtttctcttttttttttggtgttgtccctttacttttggttttaaaggtaatttgtctccctaagaagtcgtggattattatttgaaggtaattctttctgattcaaatgttccgggagaaggagaacacaagataatgtcatacattcgtctccaacgtaatcttcctggttttgatccaaatacacggcattgtctatatggtctggttagtgtcaacccataatgtacttgttttattttcttaaataaggtgttggtgcttcgtccaaaaatttgcacattacatttgccaactgtatattattatgcaggatgcagatttgataatgctctccttagctacacatgaagttcacttttccatcttaagagaggtagctgatttccacccctacgctctatattttctttgatattcagttgatcaaatgatgggtcatctttttgattccaggttatcacttaccctgggcagcaagagaaatgctttgtgtgtggacaaactggccactttgcttcagattgccctggaaaatcaggtagcaataatgcagcagctgatattccaatccacaagaaaaaatatcaggtttgtagttttccttgttatctccaagagatcttgctgcagtgaacaacagaaaattattttattttacttttggagctttctcttatttgtctgatgtaattttttttttgaaaatagttcctgaacatttgggtgttgcgagaatatctgcaatatgaattggccattccagatcctccttttatgatcaactttgagagaataatagacgactttgtattcctttgcttttttgttggtaatgacttcctacctcatatgcctacactggaaattcgagaggtatgtacatggtatagctttttattccttaaaggattggctgttcgtaaagtgatggttgtgtagattagaaaatatagtaatagccttgtggaagataaatatcctgtttcatgctctacaataccactgaggcactgaaaaattagtctgtttctttaaaccaagtttctttgttacttcagggggctataaacttgctgatgcatgtttacagaaaggagtttacagctatgggaggctatctaacagattctggtgaggtaagttattgtttctttttttaatcttctcccacatttcattctcgcttgacattgctcaataccctattttgtatgagctgtagaaatggtcagttatcttgtggaaactattttctcgcttctatttagtctggctatatgattactaatgaaaatcggaactgcattatatctgtgggcttcatttcttttaatcgttaacccattgacaacttctttttttctccattgataggttttactagatagggtagagcatttcattcaagctgtcgctgtaaatgaagataaaatatttcagaaaagaacgcggataaaacaggtaatcttaacctttaccctattttcctacacaaccctttggaggctttaggaactgtgctcacacccttatgaggctaatggtgtgttgatgttggtatcagtcaatggataacaatgaagaaatgaagcaaagatctagaagggacccatctgaggtaccacctgaaccgatagacgataaggtaaaatagtttactgcaaagaactcttatatgtgcctgtgagctggagagattcttatatatttattgaaaaggaaattagtgtaggaggaacgactaaaactgctctttcattttgggtagattaaattaggagaaccaggctacaaggagaggtattatgctgagaaatttagtacgaccaatccagaggaaactgagcaaatcaaacaagacatggtgagttttacgtcaattaacgctctctgtcactcttctgaatctctaatgatttgttataataagtctaaagttggaaaaaggtatcttttggcgatgtgctactttgattatgtgtgcatttagactttttaacatcctattaactccctcatgttgaaaatttctgcccacggaatttagctcttaatattttcacagttctttttttttcttttatggaggcttatagcgcgatatgaactacttcattgaagtgactctcaaaacagtttggaattggatgaacaataactagttatctattctgatcattcattacctattttcgttgtaggtactgaaatatgttgaaggtttgtgctgggtttgccggtactactaccaaggtgtatgctcttggcaatggtaagtctgactttacaaaaacttaatgtcatgttattcccatgagagccatgcattctaacaaaaacttggtctcttctcgggaaagaccttgatgttgatgtcacttactgagaaatactaagctgcaaattttaaatgtaattttgtgttttcttacactttcttctaattgtttcaggttttacccataccattatgctccatttgcttcagatcttaagaatctacctgatttagaaatcacattttttattggagagccctttaaaccttttgaccagttaatgggaaccctgccggctgcaaggttcttccctttttttggaaccttaattattaatattgtgggcctctatggtgatgtaaattgtgtttcttggttgttgcagctcaaatgcgctgcctggagaataccggaagttgatgactgatccctcatccccgatacttaaattttaccctgctggtaattgttgatactgaatttgtattgttgttttcctatatattcatgctggatagttgacactcataagctatcattctatttatagattttgagcttgacatgaatggaaagcgcttcgcctggcaggttcatggttgtttgtattctttggatatagtattgatgttatcatgctgagactgtgtattttgttcttcagctgttgatgttgtgtgttttgcgcttgttgttatctagggtattgcaaaacttcctttcattgaggagaaattattgttagctgctacaaggaagcttgaagaaactctaacggtttgaactgaaccttgactcgaaatgcataatgtttgatcaaaactttattctctccaaaggaaactataattctttctgaaagatgattcttggacctgcaggatggtttatttgctagttataaagtagttttatagcttagattgctctagccatcagttttcatggcagtttatgattaactacaatatggatccatctttcaggtggaagaacaacaacgcaacagtgtgatgcttgatttgttatacgtacaccccgctcatccactgggccagcggattctgcagtactatcatttctaccagcacatgccaccacatgaatgtcttccatggatgattgacccgaattctaggtttgtttttattggctaaattgataactaacaagctgtgtgtactctggatatcgagcttgttcttaatcatcaattcgtttgtgtttgttgcagccaaggaatgaacggttttctttggtttagtgaaaggaatggtttccaaactagagtggattcccctgtcaatggtttgccatgcattgagcagaaccgagccttgtgcgtttcttgtacttcacaagttagtgttcatgaaattgatgaagtcttgatttactcacttatattttcctctacattttccagaaatgttacatacctgtgtcctgccaaacactctcatatctcagaaccacccagaggagcgatcattccggacaaggtatgattcctcaaaggaattcgtcatgaacagtagtccatctctactgcatcaaaataaatagaaacttaggtttttatagcgtttatgagaatctacttgcttgcatctcaaatgtctatgttttcttaccagtctatttatctgctgaatattcattgcaaaacaagtgttcttatgaaagttcaattggcagatcttgacgtcagtagatataaagccatttcctcctttatggcatgaagacaatagcaaccgacgccgacaagcccgagataggtaaggagattttactgcagactgatattttgaagatcatagtgtgaattatgttaaaagaaccatatggtatatctattgtgcaggccgcaagtggttggagctatagcagggccttctctgggagaagcagctcatcgtctgatcaaaaacacccttaacatgaaatcctctactggtgcggcttcgggattaattgacccaaacggatactaccgaaatgtaccgggtaattattcttatggtggagtcaatagaccaagagcaccgggtccgtctccttaccggaaagcttatgacgatgactcgagctattactatggaaagtataacaatagcacacagggaacgtttaacaatggtccaagatatccttatccatcaaatgggtcacaagactataaccggaactacaactccaagatagtagcagaacagcacaaccgtggtggacttggagctggaatgtctggtttatcgatagaagataacggcagaagcaaacaattgtattcaagttatacagaagctgctaatgcaaatttgaacccgttgccatcaccccccacccaatggatcggtacacagccaggtggtaattttgtcggtggatactatagggatggagttgggtacagtgaaacaaatggaaaatcggtaaagaaagtaatttaccaggccaagacacaaccaagtcatcgaggtgcaaacttgtgagatattagttgaggcgcataaggaggttcttctgtacaggtgagttcctttcttttgctgcaatggtcttatgtatggtagcatcatctgccatcagttctcttaaatttctggctaaaggcaaatgtataatgtacctttggacatttcttttgttatatatacttattacattaaacttgtttagagtccttaagagatctcttggcaattggatgttattcgactctctgtttcttctaccgtgtttcgctattggttttaac

SEQ ID NO: 3 - protein, 966 aa, Glycine max
MGVPAFYRWLADRYPLSIADVVEEEPSVGSGAVDVSKPNPNGMEFDNLYLDMNGIIHPCFHPDGKSAPTTYEDVFKSIFDYIDHIFSLVRPRKLLYLAIDGVAPRAKMNQQRSRRFRAAKDAAEKDAAEAEAEIERLREEFEGEMKLLSSKVKPETYDSNVITPGTPFMGVLSVALQYYIQTRLNYNPGWRNTKVILSDSNVPGEGEHKIMDYIRLQRNLPGFNPNTRHCLYGLDADLIMLSLATHEVHFSILREVITFPGQQEKCFQCGQVGHFAAECRGKPGEKAEDWNPVDDTPIHKKKYQFLNIWVLREYLQYEMEIPNPPFEIDFERIVDDFVFLCFFVGNDFLPHMPTLEIREGAVNLLMHIYRKEFTAMGGYLTEAGEVFLERVEHFIQSVAVHEDQIFLKRVRIQQATEINEEMKARARGEMPGEPRASVLDKVKLGEPGYKERYYAEKFGALDLEKIEKIKKDTVLKYVEGLCWVCRYYYQGVCSWQWYYPYHYAPFASDLKDLADLEITFFLGEPFKPFDQLMGTLPASSSSALPEKYRDLMIDPSSPILQFYPADFEIDMNGKRFAWQGVAKLPFIDEKKLLSATSKLEATLTEEEQLRNSKMLDLLYVSSAHNLAPHILSYHQYSCQLPLHERPALPIDPRASSDGMNGYLWLYERNVLRTTVSSPIKGLQDIEFNQVLNITYLNPRKHRHIPKPPDGVLMPKKILKAIDIKPFPVLWHEDNSGRRQGRERQQVHGAMAGPQLGEAAHRLVKNSLNIKSNNMPYGSPEQFPGHHAMNRLRSAGPSGSGKYYGEDTSGYYGQHYNHQGIITRPRYPVSSNGGHNDKQNFRIQDRSYHHDQYYNGTTGFYTMAMEEGARARPYAVPLPRTPAVTLSRPPNSGSTTNGQHQFVQNMGPPIPPPNWITRVPDTNGIYAGHQETALGGTYDKQNMKVYQPIQVLQWSMRGQQLCDTVRQ

SEQ ID NO: 4 - DNA, 11880 bp, Glycine max
aaggcggccaccgcagccgcccgtacattaaacggcaacacagatctcctctccatctcttttttatttttgttttattttttcccactctttctcctcgtttccgaaccggaccgaaccgagccggaaacatgggagttccagcgttctaccggtggctcgcggatcggtacccactctcgatcgccgacgtcgtggaggaggagccatctgtcggctccggcgccgttgatgtgtctaaacctaatccaaatggcatggagttcgacaacttgtacttggacatgaacggcatcattcacccctgttttcaccctgatggcaaggtaaccatatgcttcgcttcacctcactttcattctattcacttttttctcgtgactcagaatattgcaaccggttaacctattattctgttaatagtatcaacattttcttcgttgtttgagaaaaaagccattatgtacaatctaatagaaataagtaaagagagcggaaaataatgacagtgaacttctgatatttctgcttcaacttctcatttcgagtatttgtatggatttctttattggatttagatatattgagatcgtatgttcctgaattggttgtcgaaaactcgttctagaatttgttgcattgttaaactgagttcctggtgctttttgaagaaatggcatcatacacgattatgtgagtagtgtgaaaccttatacaaagctgactagactgtggcaattgggatctgtaatttctttattttgcgtctaaaaggaagtgattttttgtaaatttacatttctattctgctaagtgcttgttgttgattaatctttaaaatgcagtcagcacctactacctacgaagatgtattcaaatcaatctttgactatattgaccatatattttcattggttcgcccaaggaagcttctttatcttgcaattggtatgttgctttttcttgttattgtatctctcgtctatggaacaccaagcatttcccaatactttatcttacttgtagatgatgctcatggaagaaataagattgacaccattcctctttgtgagcaataagcagaaaattaggatggcaatgatttataatttctattttgtttttagtaatgtcccaaaagcatgttcattgtttggatttgagaaatgcatgaatcatatcctatatattttaagagtagtcataatgccataccagatacatatcatatttgtgcgtcatagtttctcatcgtggaggatttgcccaactgctcctttaagttataaatccatttggttcctaacttctcattatgccttctgttttttttcgatgattagtaacttttggcacactcctgtatggttgtacattcaaaatttagtgcaaatattttcaagtaaaagactcagataaatgtttgtaaaacagaatagccagtaaggaaagttagaacttttaaatatccaatttttggaagcctgtaatttttttgtctgtaatgttatgttattgtatgtttgatttaactttgaatcgcttcctcattcctaccatttctttccatatgtacttttctcgccccttaagacggtgttgcaccaagagccaaaatgaatcagcaacgttcacggcgttttcgagccgcaaaagatgcagctgagaaagacgcagctgaggctgtaagaatatgaacatgctcctgtttacaatgatctcctgctatttctgttacaaagtttgaatacaatgtgatgttgtaggaagctgaaattgaaagacttagggaagaatttgagggtgagatgaaactcttgtcttctaaagtcaagccagagacttatgactcaaatgtcattacccctggcactccatttatgggagtgctgtcagtggctcttcagtattatatacagactagattgaactacaaccctggctggcggaatacaaaggttgaccttatcctatttattctttgcccccttgatccattcaatattggatattccttacttgtggagtaatcctgtttattttctgttcccatattgtacatatatctggttttcagaatattttgtctctatatatttatttatggagctgcatttggtattttttgaaggttatactttctgattcaaatgttcctggtgagggagaacataaaataatggattacatacgattacagcgtaaccttcctgggtttaatccaaatactcgtcactgtttgtatggattggtgagtttttgtctgattttgaaattttcaatttatccgaggttggtgtatttaattatatatcttttccttgatttttatatggtgtgataacattgatgtgatctttaattcaggatgctgatttgattatgttgtccttagccacacatgaggttcatttctcaatattgagggaggttaggttctattttatgaatagtcatggtgattttagttcattttcaaagagaaagatatcaaatatctgaagttcctttttatatattatatggatgatccgatccgtaaggaccatgattgggaattttttttatcatctttctccaagaaagaggcgaaacataatcaaatgtcagaaaacttggagaaaatgacaaatattttttagttttaactctatggtatagaagaaatagaaaaggatcatataaaagttttttaaatttaaaacggatcagtacacctggtctacgaatatattctaatcccaatagattgacctgatgattaattacatgtatgaacaggtaatcactttccctggacaacaggagaagtgttttcagtgtggccaagttggccattttgcagctgaatgtcgtggtaagccaggtgagaaagctgaggactggaatccagttgatgataccccaattcacaagaagaaatatcaggttggatattggaatccagcatgttaggctgaatattcattttcttctgttactttttaatttcaaactaaaaccttgcatgtacagtttcttaacatctgggtgctgcgtgaatatttgcaatatgaaatggagattcccaaccctccttttgagattgactttgaacgcattgtggatgattttgtatttctatgcttctttgttggcaatgactttcttccacatatgccaacattagagatccgtgaggtaagcatctgggaggcaccatgttataaatgtcaatgttgtcctctatgataaactttttactgtttcagggggctgtaaatttattgatgcatatttatagaaaagaattcaccgccatgggtggttatcttactgaagcgggtgaggtaaatttcttttttttctttcttaaattcaattaagttgtgtgtgtgcctttttgtatgtttttgtaaatctgtgatgtgttctggtatgtgactgaatcttttttagcaatcatcttccttcaatttgcttgtctcatctctcaagtcacccatctggaattttgtttcttcaatgactgttgtaatatgttgaacactctaggttttcttagagagagtagaacattttattcagtctgttgctgttcatgaagatcaaatatttctgaaacgagttcggattcaacaggtattgtgatttctgtgttttgactatccctctttggaacaaataattatcattattccttcaacttctgaaccctctggagttttgcaccaatcatattcaataatcgtcagagttctacttatcttcatggttctgtatttagagatgggatatcttgcttaatgcattgtaagaaagttcttctgtcaatattgatgtggaaaaatcttcaattcattccattctgttttgcattctgtgtattttggcccattgtttctgttgaaaatttataactgaagcatcacaactttattccctcatccatgatttgtgcttcctatttgaactttggaattaagtgattttgtaagaagacttgaaacgacactgtttcgtatcatttaaagtcatattgagcatgtatggaggaactaaattggctttggctactccacagattaaataattataaaaggggtataaactgtagctgaggcatgaatttgatatggctgtatcatacttaatttgggagtggagttctcctctaatgttccggtgaataaaggaaatgacttgtaagtagacttcagtggttaggcaaattttggtcagcatgtacagaattatcgaggatctttatccaatgaagttccagaggacaattccttaatccttgttccactgtactagggtttaagtatagtgtctccctctaatacgtaactctcaactttggtggttacctgttactgacgtgcaagttcccaagccaacaccaacatgatactattgtatttgttaccatttgttttcttttctctcatatgcactgttctgaaacaaattctgatgtggatgtattgtgacattagactttacatccgggagatgtcattcacaagtccgtctaattagtttcatgaactgatatgattgagctatatatttctttaatatagttttaaactttatttcctgatatgcaatacttgtgatttgattgtttttcaggctacagaaattaatgaagagatgaaagctagggcaagaggagagatgcctggagaaccacgggcatcagttttggacaaggtttggtaacaagattttttgcccaaatgttttaatcatttcgcttgaatttagtattttctgcttaattttattggaccctaaaccatagtaattctagtggcttcccaaacatacattgagggcagcatgtctgtgagtttttgtgctctgctggtctgtgggtgatcagtgatgcggttcccacttcctgccacttaatttcagtttttctcatattaaaattgttgtcaggaagccttgtaaagcttgtagtaactttaaaaacttacgtctgttatgtattttgagaagatacttcaatagtagttgtgcgtgttattttgttgttactattctttgtttcatttttgtatgtatacatatattatattattctatataattttatatataacaggtaaactctacctcccttgtctgatcctaggtgggatctattgttttggctacctttggaagtaaggccgcagagctcttgatgttacttatagtttgccttgttgcatttatggctaaccagttatggggcaatctgataatttttagtagccatgttgaagggtttatacttgtacttctgttttatcccccattgttatataaaacatgaactgcatgggcagtgctggtgccggaaccaacttcatcatctgcctacacaaaaagagtttgtgtcagtgtgtgtctgttgatatcaatttgaagttaattgctccctaatttaactgtttggatgcatgaagaaaatgttttctttgtctctcttctgaaacaacttggttacacttttacttttctcatgtgttttcttttagtgggaacaactgatgataactctgactttcccaactagaatttgtctttctaaaattaaatgttccttttgtcagttgaaagtagctaaaagatcctgggttagaaggattcataatttttttggttagatacaattcatgtaagagtgatccctgaatagggacacttggccaatgtcagaccacatgaaatccaatagtgagaaaatttataaactacatattgtttgtgattttttcaggttaagttaggagagcctggatacaaggaaaggtattatgctgaaaaatttggtgcattggatctggagaagattgaaaaaatcaagaaagatacagtatgtcctgtcagtattcttggaattgtttactgtcaccttcctttctatacacattcatggcttaaaatggaaacaatttcaaaagtacaaattcattgctaagtttttcaatactaaaagtcaagtgtcgtattatcagtgaaaatgattgtatacagggcctgtttactttgtgaaaatgttttctattttcactttcaattacaagaatgttgccttattttcacatggtttcctgttttcaaaagtttttttttcactatttcttatataaactttttaaaacaggaaacattgtacaaaaaggtgacatttttgtaattaaaagtgaaagcagaaaacaattttctcaaaccaaagaacccttttctttttaaatgatgttagacacatgctgttagtctgtcttgaactcttgatcttactatctgactgttacacaagagggagtgtgctcgtatagttcctcaatctgttatgctcatgagagaataaagacctcacactggttgaaaatttatgttatgtttggctcacgaattgataggtggtttgttggattttatttgttttttccttcttgtggatgactattgtagccaacttcaataatctatggctttcatattcatatatattgcattagaagatagctgatagtcttggttttccttgtaggtcttaaaatatgtagaaggcctttgttgggtttgtcggtactactaccagggtgtctgctcatggcaatggtttgtcgtgcttttactagttaatagattacatttgaacccattcctgctattttctgttactggattgataagctggtacatttatgttggaagttgccttagttgtggccacccttagcctgccttaattgcagcctcaaggtggtgtttagttccatgttgactagatatataattaaatatagcttatatatataggcttgggtaattgtcatcttacaagccatttttgtggggtttgagttaggcctttttttttttgctcagcaaaaataatatattataaaatagtaccagaggtactaaataatacagctatagggacatgtcatacgaagttaggatacagaaatacaaaacacaaactgaaaaacccacagtacaataattgagcaagaaactagactatattcctatgctggtgtcctaagaaatgcagctttgaggtttgaggaccactgattgaaatgcaaagagaagtctttttccaagcatcttagccatgtccacataagaaacactgcatcttccattaatttatgagcgtcaaaagtagcattagagaagatgatgctattcctatgcttccaaatagaaaaagtcagtgctaaccaccaccacagccatctgatttcctgcattccttcaacaaccccatggacatgctgaataaagtgctgacccgggtgatatgggaaaacccccaccatatttacccaagattgagactcccaccaaagaggattaattttactgcagtgaaagaataaatgactcgcggtttgagttaggccttaagccccaaattctaagaatttacatgcatgatactataatgtaaccagtttaggtgaaaaatgcacttttaaacttatgctgtgaaaatatccccacttttggtccctcaaaggatcaattgtctggaactatggatgcttagttgacttcatgtgattgttaacctgataatatcctagggcaatgggtgttttgaaaaatagatttactatgtctgttgaatttagagaggagactttagagaaaaaaagagaacttagaatgaaacaacttatttagtagattgtgtagtacataggttctgcatatcatccataatatagtttcttaatagacttagtggagagtatttcctaggctactattaattaagtgcaacttcctagacactaatacccttgggttctaaatgcattttatcatgcgatacatgaacatacaatttcctaagtaattaaataacattatttccaacaacgggattagtaaagttttgtcggttaactaaacattcaattatagtgtgccaaaagcaatgggtacaagtggcttttcatagtagaatgggtacaagtgacttttcatcattttgtcgcttgagtatgacagttattcctcatttgtgaataacactttcttgtgttccttctccacattgtggtatgatgatccaaaaaatttatgatactttgcatgaagggtgataagacaaataaaatataaagggttgattattttctttcaagattatcttgctccacatatgttcttttagcaagtttctcctgtgccttcatattctctttgaatattgagctttcattaaattctgttattattccaaagatggttgcaagcaggaacttctaatcatgattttataaggagtgtaacaagatataatatggactggatgccttgagggctgggatacaggctttgatgtttgagtggctttaagatgattgacataaaattctgagactattcttcattttggtggagtctatcacttatgactattcaattttttcattattgtttgttcattcaggtactacccataccattatgctccttttgcttctgatcttaaagatctagctgatctggagataactttttttttgggagaaccatttaaaccatttgatcagctaatggggaccctacccgcttcaaggttttttcatctctcctgtcctcagatctattatatcatgcatcctgtgctgatgctggttaagttcttgttacagttcaagtgcacttcctgaaaaatatagggatttgatgattgatccttcatcacctattcttcaattctaccctgctggtgagatcacttgtctgttattgacatattttgctatatcttatagagtttctagaattacttgtatcactttttgacagattttgagattgatatgaatggaaaacgatttgcatggcaggtaatatagtttttgtattgtggtttgtgtgtgtgttttaatgttttattaacactttataatgaagggggattaaggttactgttggaaatctttcagggtgttgccaagttgccattcattgatgagaagaaattgctttctgccacaagcaagcttgaagccacattaacggtctggaaatgtttcttatttttacttttctgggggaggggtgttggtttttacgatatttagttctggtctattacactggtgactaactatataagattagtggtccaaaattgttggccttcaaattgcatcaaagaataatttgtcaaagaagtttactgctatggtttatcaaaatagaagttgttgaatagatctgttgagtgatacccctctgttaatgctgtttctttttgcacaattatccccaactctttgctgtgcaattgctggaatttttatttttattttggtttgagctggaaatatttttagtccactagaagtttacagcagttaatttgaataatatttttgcatctgggtgctcaatttcattcacatttgttatgtgcaggaggaagaacagctccgaaatagcaagatgcttgatttgctttatgtcagcagtgcgcataatttggctccacatatcttatcatatcatcaatattcttgtcaattgcctctacatgaaagacctgctctaccaattgacccaagagctaggttagtgaatatgtttggtaatggtcaccagtcgttatcataaatatgtttggtaattgtcatcagtcatgtgtgccttcttttctttgttagcagtgatggtatgaatggatatctctggttgtacgaaaggaatgtgctcagaaccactgtatcttctcccatcaagggattgcaagacattgagtttaatcaagttttgtaagttttgttctttagacttgcctaatattttaaaatcattgaatgaactcatgttgagtgtttcaaatttcagaaatattacttatctcaatcctcgtaaacatagacatattccaaaacctccggatggtgtactaatgccaaaaaaggttggtatagacttctcttagcatttgacattatcaatggatacatcctgtagaaggagcttcatgattcatttgtttaggcttaaggggggcatttgaaagagttaatgtggagttgatttagaccatgaaaatagcttgactcccgtaaattctttttagcttatttcaataagctttttagtctagcttatcaaaataagctctttttagcttattcccataagtttcatgggcaaatttatggcataagcttttatgtgataggagcttattaaacaaacacctaattaagctttacctaagcacaccctaacatgtgtttaagcccatcccgatagttttgatccattaagtggtgagatcattgccacatggcataacctgatatgccacatcatcacttaacagaatacaactaccagcatggactacaaacttgcatttccatgaaatcaatgatctaatatatttttttagggaccaaaaatgaaaattggctaatttttcagggatgaaaaatttacttaaccccatttatttatgtttagaatcagattctcgcgtcaataacatagctcagatagttgccatgactttattcatttaatcaaatattatactttcgttttagttttttcaaagaaaaattatactccttgttttcttctatggatagatctcatttacttttgacattggttatgtttgcagatattaaaagccattgacattaaaccttttcctgtgttatggcacgaagacaatagtggtcggcgacaaggcagggaaaggtgagaatctattcctgaggttgctatattggtgtagtataattttcatgttctttctgtctggtatacaatccacaactgtttaaatttaatgttcaatgtggtagtggtactgttattactcaccaccttatgtggacaaattttttttgaggaaattgtcaatattaagtaattgtcctgtggtgaatgtagagaattccatgttgagtgggttgataagttctcaatgtctttcaaatttagcacatgaagttttttatgcatggaatctggtatttatgtgagtgataagacatatactttaaaagagctcttacctttcttgtgtgatgcatgtgacagacaacaagtacatggagcaatggctggccctcaactgggagaagcagcacatcgtcttgtcaagaactcccttaatattaaatcaaataacatgccgtatggatcaccggagcagtttccaggccatcatgcaatgaaccggctcagatcagctggaccttctggatctgggaaatattatggtgaggacacaagcggttattacggacaacattacaatcaccaggggataataactaggcctagatatccagtctcatctaatggtggacataatgacaaacagaattttaggatacaggataggtcatatcatcatgatcaatactacaacgggacaactgggttttataccatggcaatggaggaaggtgcaagagctaggccatatgcagtgcctttgccaaggacacctgcagtgacgttatcgaggcctcccaattcagggtcaacaactaatggacaacaccaatttgtgcagaacatgggtcctcctataccacctccaaattggattactagagtaccggatacgaatggaatatatgctgggcatcaagaaactgcattggggggaacttatgataagcagaacatgaaggtgtaccaagttaaaactcgtcaaccccaagatatgccagagtacgggaatcaatgatgactgttgtggattttattcgccttgtgacgaagtgccctcttccattcctcgggctgtttctcaaatcggagcccatccaagttctacaatggagtatgagggggcaacaattgtgtgataccgtaaggcaatag

SEQ ID NO: 5 - protein, 962 aa, Malus domestica
  misc_feature (148)..(148): Xaa can be any naturally occurring amino acid
  misc_feature (180)..(180): Xaa can be any naturally occurring amino acid
  misc_feature (218)..(218): Xaa can be any naturally occurring amino acid
  misc_feature (608)..(608): Xaa can be any naturally occurring amino acid
  misc_feature (645)..(645): Xaa can be any naturally occurring amino acid
  misc_feature (941)..(941): Xaa can be any naturally occurring amino acid
MGVPAFYRWLADRYPQSIADVVEEHAREDSNGVPVPVDVSGPNPNGSEFDNLYLDMNGIIHPCFHPDGKPAPATYDDVFKSIFDYIDHLFTLVRPRKLLFLAIDGVAPRAKMNQQRSRRYRASKDAAEAEAEEERLKKEFEMEGTSLXSKEKPETLDSNVITPGTQFMAVLSVALQYYIXSRLNHNPGWRSTKVILSDANVPGEGEHKIMSYIRLQRXLPGFDPNTRHCLYGLDADLIMLALATHEVHFSILREVITFPGQQEKCFLCGQVGHLAAECRGISDDNGNVVDDTPIHQKKYQFLNIWVLREYLQYELDIPNPPFQLNFERIVDDFVFMCFFVGNDFLPHMPTLEIREGAINLLMHIYRTEFTAMGGYLTDAGEVLLDRVEHFIQSVAVFESQIFQKRVRIQQAIENNEERHRARRETSAELPAPVMDKVKLGEPGYTERYYAEKFQVSTPEEIDKVKKDLVLKYVEGLCWVCRYYYQGVCSWIWYYPYHYAPFASDLKDLNELEITFFLGEPFKPFDQLMGTLPAASSSALPEKYRNLMTDPRSPIHNFYPADFEIDMNGKRFAWQGVAKLPFINERKLVTETRKLESTLTEEEQARNSXMLDLLYVYPSHPLAAQISMYYQQFYQTPLHERFLWRXDPIASGGMNGFLGLSERNGLRYVIPSPIRGYPDLDCNQVLNITYLNPADHKHIPEPPKGVILPRKVVRPTDIKPFPTLWHDEGPRRQGRERPQIPGAIAGPMLGEAAHRLVKNTLNIKPNGSSPGLWDQPPLRNSGNYAGNYPVNRPRPAGPSGYERVFREEATYGNSFNPQVMMARPRIPASNGMQGDRQNFRAQERVQHQEQNFRTQDRVQDQEQYNLRSVMSSLTVQDSGRTRSPAAAPPGMPNSGYQANRKHQFVQNMGALPSPPTKWISKEPTGNGGLHVRKQETGYGGAXEQQQVKKVYQVKTRAPQDVKP

SEQ ID NO: 6 - DNA, 8757 bp, Malus domestica
atgggcgttcctgcgttttaccgctggctagcggatcggtacccacagtccatcgccgacgtcgtggaggagcatgccagagaggactccaatggcgtccctgtgccagtcgacgtctccggaccgaaccctaatgggtccgagttcgacaacttgtaccttgacatgaacggcattatacatccctgttttcatcccgatgggaaggttcgctctctctytttcctctgcaatttccaatttccgttcgttttgttgtaaaatgttgattttggtgctctcagaggtagaatttagcttaatttgtgtctaatggcaattttttgttgtacaaaactatgggttttggaggtgtttgtttgggttgcttcaacaatttcgtactttttgttgcagtcggtgagaattgagttccagattgattgtttgcatgttatttctgtcgtgtttgcgtttgatgatcgaagtgattgattttggtagtctttagaggaaaaatttagtcttcagttatacacatatacatatacatatgtggcttgacaatacttaaatttagtgcctttttcttgtgggaagtacgtgactccgttgtttatcttgcatatgttgacttaaatattgacttggactgtcagattgggtaaaagttatcttgttgtttatgtgggtgtttgaaatgtgggsatttaccaggttattatgtgctttgagtgacctctcgatgaggaaatgcctttgcttaaagttgtattgtgatgacatgttaattttgtgtgtgtgtgcgtgtgtgcagcctgccccygctacctatgatgatgtattcaaatcaatatttgattacattgatcatctattcacattggttcgcccaagaaagcttctttttttggcaattggtaagatttaacttaagacatgttaagtagtttatctataatgttttttcttgcgctaaatttagtacttcatatatttctaatcttgtagatggggttgctccaagagccaaaatgaatcagcaacgttctcgacgttacagagcttcaaaagatgcggctgaagcggtaacatcttactcactgtgcataattagttcctatccagggaggcagtgaacttattgttgtttttcttaaatccttatgttaggaagctgaagaggaaagacttaagaaagaatttgagatggaggggacatctttgscttctaaagagaaacctgaaactttagactcaaatgtcataaccccgggcactcaatttatggcagtgctctcggtggcacttcaatattacatacawagtaggttgaaccacaaccctggttggcggtctacgaaggttggtttgacatcattgcctgtattgatttgcaattgcctccctcagtcccttcaagatcagatggttctctgtctrtggagtaatccattctctatatttttaccatgtacttctggttttcagggtactttgtctctctaaatttatttatggggctgcattgggtggttatttgaaggttatactttctgatgcaaatgttcctggcgagggagagcacaaaataatgtcatacatccgattgcaacgcarcctccctggttttgatcctaacactcgacaytgtctttatggtttggtgagtgtttattatttaggcctttttttgcgagtcttttatttgctgttatcattatcaggcttttgtaaataatttttttttgccgtgtgtgtatgcatgttttatgtcctttgaatgggaagccattttgtatttcctttgctgcattgtttagttctaaaatgggwatttggatcatcataatcaggatgcagatctaattatgctggctttagctacacatgaggttcacttctcaatattaagagaggtagttgctctttcttcagaacattttctaagttgtttattacaagtggttgatcttatagattgtttaccaaaattgtacaggtgattacttttccaggtcaacaagaaaaatgttttctttgtggacaagtkggtcatctggcagctgagtgtcgtggtatatctgatgataatggaaacgtggttgatgatacrccaattcaccagaagaaatatcaggttgaagaaatggatcctgcttctcgttgctaccctttttgaattcctcttgatatcattctcatattattcctaaattatattttgtttgtgcagttcctcaacatatgggtattgcgtgaatatttgcaatatgagctggatattcccaaccctccttttcagctcaattttgaaaggatagtggatgattttgtttttatgtgcttttttgttggcaatgactttcttccacatatgccaacattggagatcagagaggtacgttattatagctagcagtctgttgctttgattttcagaagtytgccaattttaggggtttaggtactaagtttattrcattctcgtcaaattaaagtgtctaagatttgttaacttttaccgtatcaaacacatgattgttcactgctccttctattggttgttcacttttcttttttacttgatgaaaggataaagttttggccctttcacatgcaagcccttgataacattgcactttagataaaagcacttcaaacgcaatgctttagaaaaagacctctgactttgtaatggaagaaggccaccaaaggaatttcacagctaataatcaattaaagcatgacaccaaaggaatttcacagctaataatcattaaagcatgaaatatgtatrgattaggttttttttccctcaagttgatcttttgaggttttttgtataacttgtagttgttggkgtattttctaatttactattaactttttactgcctcagggtgctattaatttattgatgcatatttatagaacggagttcactgctatgggaggttatcttactgatgcgggtgaggtaaattgctcttctttacatgtctgttactcttgtatctcattgcatttgccataaatgcgttctgatgtgtcaattgcagtttcttatgtatgatgtttgtaatgggtgctacatcaagtttctcttgttttttatatttgaaaaagtatcaactgtttggatgttaacattttttaagtgagtgaaagatcttaaatgcacccatgaatgagggttaatgaatgccatttctatcattgctactataacaatattgggaagacctacttttgtaatgtaggagactggtgcacttaaatcttattatcgatctaatacattagctcaccgtagtagactgtaactttaattattggagagccggtcctatagatctgtggatgactgcagtattgcgtgtaaatgggatacaatgcctgatattgaaacacaatcaagatagcaatgaaaggaaagagtggaacagggttattcatatttgtcagatgtgttcattatgtgtaaaatattttaatctctctctctcacccccttgcacttaaaaatgtggatgcttgctctgttattgcttttgtttgttttgaatatgtttctatgaagaacttgtgcataacattttaatatattatcttttggcaaaaatctttttgatctatatttaaccctagttgattttagtgratattggtaccatgcatgtgcttatccatttgagtttctctcacaggtattgctagacagagtagagcattttattcagtctgttgctgtttttgaaagccaaatctttcaaaagcgggttcgaattcagcaggtcttcckcttctttctgatttaatgtgtactatcagttcatttgtttgtcaatttactggagcatcatgaaaccaagggatgatgacaaatttaagcatgataaagaatttactcctttcactgcaaaattgttgttcgtccattagtttcttagtcttatttttgtgggaagttttttgtcaatactgatacctagtggactcttttaagtttcttttggtgattacttgattacaacggaattcatctggtcttaaatattttcccattagaatgagtgagatgtttggttaaacaaaaatgaaaacttcctatgagatgtaaatgatggtagttaaataagcctttctagcaaaaggcaagaaatttrtaggtataagcttttgtaggggcrtttgtkcacttgatatttctttgatgaaaggaaatttgaacacgatgataaatcataattatgtagtgacttatgtgttatgtcctggcsttatgtggatccatttgacagaaaagttaatccactcacatttagcactatgaatttcgctwcatgatrtgtgaaccaaaggcttagagcctttcttttataaccatatttgagccatggaagtgaacttttgttctgggggttaattgtgggattatataggtcacttaatcatgctaygggtatccaaaactatttattggtyrgaaatttgctcctttagtcaatcatacatgtctttcttcatrctgatgatgattatattgcaggctattgagaataatgaagagagacatagagctagaagagaaacttcagcagagctaccggctccggtgatggayaaggtctgcaatataattgcaactgtttgttcaaattttaaaataatacagtgataaaataacttagctttttgtgccatctattgtacatttgttcactcatttcactggaaagtcatgaagagactgaaagcttctttgaagggcaggccaagtaattaaggaatgtatggtgtttgtgcgtctgattttattttgtttctcaggttaaattgggagagcctggatatacagaaaggtattatgctgagaaatttcaagtatcaacaccagaagagattgataaagttaaaaaggacttggtaagctctctcaggcgaracattttagttatattatttggctgtggatatggttttgtttttggatattacactacttatttttgcttttcatgtgaacggatatctttattgaatcatctgccatatatattttggattgaacctkggaggtttaagctgtgcgtggggtctgtagctctacccatctatagttggctttttttttcttwggagaaaaaaaaawagtgatcgaggaaaaaggttaaawcactgtgatcaggtctgtacttagstatgaacagtcggtaatttaggagatcagaagatttcatatgttttgagtttaggagattcttgttgggttcagttgctgccttcatgccattctaatgaacttaatctaatttaaaagcttttcgtctttttgctgttgtttcttcctggtttgataggacaagtactaagttactctatgctaaaatatttttgtttctttgcaggttttaaaatatgtagaaggcttatgttgggtttgccgatattattaccaaggagtctgctcatggatatggttagtttgactttaaccttttgcttcttttacctgctttgcctcatcttattgctgatctggaggtctgactwctctatctggaagttggatgggtgacatgttgagccaccgtgttgactggattgatatccagttcagtttcaataactttgcttgtatctgtaatcgttttttttttcaggtattatccataccattatgctccttttgcttctgatctaaaagatttaaatgaactggaaataacatttttcttgggagagccatttaagcccttcgatcagctaatgggaaccctaccagctgcaaggtctctctcatttgtggctttgaatttattcaaataagttatttatatatagaagatttcatcttctagatatgataatgctcagtatttctattttacagctctagtgctcttccggaaaaatacagaaacttaatgactgatccaagatcacctatccataatttctatcctgctggtaataattgtctagttgtatggatctttttatactgtgaagttgtatcctatctgataaatattttccttttgaaatatctcactccagattttgaaattgacatgaatggcaagcgctttgcatggcaggtatgatcttctgtagtgttgttgttttwctttattttttaaatttgtgtttgtttttccttttggaggggtaaactaatggtagwttgatcactttatgtgtctgtgtgtgtgtgtgtgtttggaagtcagggtgttgccaaattgccgttcattaacgagaggaaattggttactgagacaaggaagcttgaaagcactttaacggtatggaagaatattcattttcttcctaataatttttgcatcaattatacttcatgttcatatgtttacttgtcaaatttataccccttaaaatggattttgtataccaatacttatgtaaagcattggtttttacaagttctaggttagcttgcaagagttatatttcagtttttacaatcagggtttttagggaaacatatggcaccatgttctgatagactgaattttggggttgtgagaatgacatgagatattaatccatcctatcatgcctatctmttctaggtctgaacaaatagtattgaaattattggctatgtrtatgtattgataatttgagattgaaatgcctgaaaagttgatggtctttggaatcatggattacatttgggtcagagtaggagttaaagatcttgtatggtgggctgcaatgagcctacgacttatctaggattctctatcttaccgtagggctattttggacatcttttaactgcaataaccacgataagttggccctcattttactactacattttgagggttcatactctttgtcatatgcaggaagaagagcaggctcggaatagcrtgatgcttgatttgctttatgtttatccttctcatcctttggcagcacaaatcagtatgtactaccaacagttttatcagacacccctacatgaaagatttttatggcggrtagacccaattgctaggtaggttattttagtagtaatttatggggactcatggtctaattgtcactgtggaatttgcttgacttgtgaccctcttgtttctttttctttttttttcagtggyggaatgaatggattccttgggttatctgagagaaatggcttaagatatgtaatcccttcccctattcgtggttatcctgaccttgattgtaaccaagtcttgtaagtagagcctctactattgttgtttaagtatttaaagagaatacagtgttttcatggtgggacttttcgtatgcagaaatattacatatcttaatccggcagatcataaacacatcccagaacctcccaagggtgtgatcctgcccagaaaggtatctggattctttaaagttcttgtccaagtcttacagaaattttttcgttgtagtccatgtcattacgttttacatggcatgtcaaatacctattgctcatacccccctgtgaagacattcgttcttcccrttaaagctgggatatgtgatatatcaggttcctgtggttggtgtttctttggttcatggtttgcatacatgaaaatattacatccttgtggctttkttcaatgtgaattttctaatgaaaaatattatgttatttctattttcttatggttaaaaaaatgtgagtagctgcatataagttgagcttttcattatcacttcatgtgttgatttatcgtcacattgctctgtgyttggagatgctaagacagttgggaatatgcttcaatttcttgtcctctttttctgttatcttttctattacacttgtttatcagggggatctaatttggattgtccttttaaatgtctttaggttgtaagacctacggacatcaaaccattccctacgttatggcacgacgaaggtcctcgacgccaaggcagagagaggtgagttgaattccatatgtccggattttacatgttttttgcttagtaacaagatctatatctgaggatatttatcttataactgcaactcggggctctggattgtggtttctgaacctgatgcgtttgttttataggccacaaatacctggagcaattgctggacccatgttgggagaagcagctcaccgcctggtcaagaacacccttaatattaaacctaatggttcatcccctggattatgggatcaaccgccattacgaaattccggaaattatgcaggaaattatccagtcaacagaccaaggccagctgggccctctgggtatgaaagggtcttccgcgaagaagcaacgtatggaaattccttcaatcctcaggtcatgatggctaggcctagaattcctgcatcaaatggtatgcaaggtgacagacagaattttagggcgcaagaaagagtacaacatcaagaacagaattttaggacacaagatagagtacaagatcaagaacagtacaacctgagatccgtgatgtckagtttaacagtrcaagacagtgggagaaccaggtcacctgcagcggcgccacctgggatgccaaattcaggataccaagcaaaccgtaagcaccagtttgtgcagaacatgggtgctcttccttcgccacctaccaagtggattagcaaagaaccaactggaaatggtggattacatgtaaggaaacaggaaactggatatggtggagcatrtgagcaacagcaggtcaagaaggtctatcaagttaagacgcgggctccccaggatgtcaaaccgtag

SEQ ID NO: 7 - protein, 1068 aa, Oryza sativa japonica
MGVPAFYRWLAEKYPMVVVDVVEEEAVEIEGVKVPVDTSKPNPNGLEFDNLYLDMNGIIHPCFHPEDRPSPTTFAEVFQCMFDYIDRLFVMVRPRKLMYMAIDGVAPRAKMNQQRSRRFRAAKDAADAAAEEERLREEFEREGRKLPPKQQSQTCDSNVITPGTEFMAVLSIALQYYIHLRLNYDPGWKQVKVILSDANVPGEGEHKIMSYIRGQRNLPGFNPNTRHCLYGLDADLIMLALATHEVHFSILREVVYTPGQQDKCFLCGQVGHLAANCEGKVKRKAGEFDEKGEAIVPKKPYQFLNIWTLREYLEYEFRMQNPPFPIDFERIVDDFIFMCFFVGNDFLPHMPTLEIREGAINLLMAVYKKEFPSMGGYLTDACTPDLNKVEHFIQAVGSYEDKIFQKRARLHQRQAERIKREKAQAKRGDDLDPHVRDDLIVPVARFQGSRLASGPVPSPYEQNGSDKNNGGKNSRARKAARVSSSGSSIAAAIVEAENDLEAQERENKEDLKTMLKDALREKSDVFNSENPEEDKIKLGEPGWRERYYEEKFGARTPGQIEEIRRDVVLKYTEGLCWVMHYYYEGVCSWQWFYPYHYAPFASDLSGLGQLNITFELGSPFKPFDQLMGVFPAASSHALPVQYRQLMTDANSPIIDFYPTDFEVDMNGKRYSWQGIAKLPFIDEARLLAEIKKVEHTLTPEEARRNSIMFNMLFVNGSHPLSPYIYSLNSKFGHLPDRERNEIKEKIDPSSSGGMNGYISLCSGDPCPPVFRSPVDGLEDIMDNQVICTIYKLPDSHKHIARPPVGVIIPKKTVEATDLKPPPVLWHEDSGRRPHDNNNRRPYENSNRQNPAGAISGRQLGEAAHRLVVNSLNARSGGQYNTPSMPYQTIMNGMPYPNGIPPRMEQPAPGWHVPGDLPNGQVPPAYASSSGHYQNDRSGPSQYGRDNHGRYPYARDNHHDSRGRVPPYHQSGGNSYQSHSAPSAGPGRYAQPPPYAGGYGRSYQPAPYGGGQQWQQQQQQPYGSYAGSGPYGGGAPPARPNSRPQQSQNRYNTLDRNSNRRPPPGHGRH

SEQ ID NO: 8 - DNA, 7877 bp, Oryza sativa japonica
atatacccccctcccctccccgcccgcgtggcctacccccttcgttagggtttcgcgccctcttccctctctcccttcccctcggccgtcggcccggctcctcgcggcggcggcggcggcggctcgctttctagggttagggttagggttccggcggcggaggaggaggagatgggtgtgccggcgttctaccggtggctggcggagaagtacccgatggtggtggtggacgtggtggaggaggaggcggtggagatcgagggcgtcaaggtgcccgtcgacacctccaagcccaaccccaacggcctcgagttcgacaacctctacctcgacatgaacgggatcattcacccctgcttccaccccgaggatagggtacgtcgatgttttttttttgggcgtttctgtttgcgtagctctggggtttgtttgggtgttcgtatggtgtccctccgctgtacaggtgatggatttgtgtatgcctgattgatgtacgacggagctgtggcagaggagatagtttggtgaaatgattttgtagcttcctgttttgtaatttacctgagcgttaaacataattttgcaaataaccacaaactgctaaattgccacatgagccatcttaggatgaggatgctgctgtagtttaatcttcaaactctgcatcgcaaatttgttaatttgcctttttgagttatgtttattttcattcggtggacgcaccagtttagactgcatgatgctactctgtcattgccctgcatttctcttcgaacaaatattttaatgtttactgattgctcaagtgctttctgatttctctgctgaccatgatttcttgctattgtgttatcttttacagccctctccgacaacattcgctgaggttttccagtgcatgtttgattatatcgataggctatttgtcatggttcgccctcggaagcttatgtatatggccattggtacgtataacatctgatgtgtaggaattctcttactacctttttccttcctaaaggattaggagtgtttagtgtcattgctcttttacctgttggtcatcatcatagaacagcttgccatactgccaactaatgcctttgttgaagttagctatatactatgacgagtcatttacatgagttttgaatcaggcataatcctcacccgtaaactatttatttggcagcatagtacctgtgcttgtaaacttttgctgtccagggtcctgttttttaccacatatgttttgttcttgtcgatggtttcttgttgatattttgtattatcttacaatggttttacagatggtgttgctcctcgtgccaagatgaaccagcaacgctctagacgattcagagctgcaaaagatgctgctgatgcggtcagtattctagtgaaattatcttcctttatgcagtgtatgataatatgtcgacatttaaattcccattcatcgacggcttcaaggggtagaattgcatttatggcttattatttttctatctccaggccgcagaagaagagaggttgcgtgaagagtttgagagggaaggcagaaaacttccaccaaaacagcaatctcaaacatgtgattctaacgttattactccaggaacagagtttatggctgttttatcgattgctttgcagtactacatccatctcaggttgaattatgatcctggatggaaacaagttaaagtaagcatggttttaagcctatcagttttgttttggcattacattctgtcagtatgttccatatgttaacttcacaaactctaaggtttacattgattggaaaggcaagtatgaaaaagtcatgtgtgttctggttaaattcttttgaaggttattctttctgatgccaatgttcctggtgaaggggaacataaaattatgtcctacattcgcggtcaacggaatctccctggatttaacccaaatactcgccattgtttgtatggcctggtatgttctataaacctattttttatatattttaattgggcaatgtttcttctgttggtcctgctaatgcatttttttatatatactcttgaatgcaggatgcagacctgatcatgttagctcttgcaacacacgaagtacatttctctatactgagagaggttagctctcctcaggacctagaatcaatgcctttttgtgtaaatacattttaccatgttatgctaactaaaagagcggattagttccttatgctttcttgtttatgttatttgttttctctctcttctgactatattttggtttcttgcaggtagtttatacacctggacagcaggacaaatgcttcttgtgtggtcaggttggacatctagcagcaaactgtgaaggaaaggttaagagaaaagctggggagtttgatgaaaaaggtgaagctattgttccaaagaaaccttaccaggtaatattgtctctttcctttgttgcaatttgtggtctaataagtacactttatgaatttgctgaggaccttgttttattttaattgctccttgtgtgacattgcagttcttgaatatatggaccctccgggagtacttagaatatgagttcagaatgcagaatccacctttcccgattgactttgaacgcatcgttgatgatttcatcttcatgtgtttttttgttggcaatgattttcttccacatatgccaacattagagattcgtgaggtttgttacctgttgtaaagctaaatgttttactggatatcttcgtttgaagctagtagctatgaaattatcagattactacttactagtaaattggatatttattttattcctgcttacagttctcttgtagatttgttattgcatgatgttaacttggcttttctgcagggagctattaacttgctaatggctgtatacaagaaggaatttccttccatgggtggctatctaactgatgcttgcacggtaattaaaaagccactcctattgaagttacccatttgtgtgtgtgtgtgtgtgtgtgtgtttgtttgcagaaagtagtattttattccaactgatccaagttggtatttcagtattaacatgtttttcatctgttacattagccggatctgaataaagttgaacactttattcaagcagttggctcctacgaggataaaatatttcagaaaagagctcgtttgcatcaggtgattttattttattttactccttagccttttcaattcccttgttatcatgttatcatctggcacctaatctttatttttactccatgccactatgcgccaaggggttcaccattagctatcagttgaaatttctgtttgtatttggcaatgtaatttccttatttgcaaagttcattgagtgtttattttgatatactggtaatttcagcctgatgtactgcgttatatcttctatgacatgttgtctataaacttccttcaaattggaggtagtgattgtgaatttgccacctgccaccaatagtccaacacactgcatttcttactaattgctccgtgtattgttttaatattacaccgtatatcttctctatgctgctttcttcttagctagccttgtaaattcacaatataacatggaaaggttcacaatgaccagataatccttgacatgtaggttagtgtttttttttttacaatacctctaaagaagttaattctacaattagcatgtgtgaacactgccaatccttgaaaagaagtaaatgctatgctccgtagaaatcacattatgttttgaacaatattgagacatttttgattcccatttagctagtaaaatcagaaaaaccagttgattgttcaagtatcttctaaactagttatttgttcaaggttttctcccccatactttcccctagaatttaaatgttacccatggaaaacttctgcatgcagcgtaccacgtccgtatggcttaggcatgttccatcattatttatccttagttatgcaaagccttctgatatgatcaacatgaactcttgagaaataaatgtttgcaacactattctatatatctggcggtcaaatattcaacaagtcattgatgttaagttcatatccttgtgtagcgtcaagctgaaaggataaaacgagaaaaggcccaagcaaaacgaggggatgacctggatccccatgtcagggatgatcttattgtacctgttgcacgcttccaagggtctcgccttgcatctggacctgtgccttcaccatatgaacaaaatggatctgacaaaaacaatgggggaaaaaatagtcgtgctcggaaagctgcacgggtatcatcgtcaggttcaagcattgcggctgctattgttgaagctgaaaatgaccttgaagcacaggtagctcatttcatctggctccaggagaatttatatttgtttttgaagaaacatttttgtgattttatctgacacttgttcctgtatctttcttaggaacgcgaaaacaaggaagatttgaagacaatgcttaaagatgcacttcgtgagaaatctgatgtttttaattctgaaaatccagaggaggacaaggttagtgtttacacacaagtcatgccactgctgtgctggaccttattatacgtttaattaaagtgtttactttccttttattttaactcatttactttctattagctatttgtttaaatgttttcaatttagtcataacattgtttgtcttctcagataaaacttggagaaccaggatggagggaaaggtactatgaagagaagtttggagcaagaacacccgggcagattgaagaaatacgcagagatgtcgtaagtgtcttgaatacctttgaattactgcattgcctgataatggtgtcatgatgatctattgctgttaatatatagttgtacaaacttccataaaagatgccatttcatcgctatttgagcatgtactcatttattatcttatatgtatgatgtgccctattcatcttcttcaggttctgaaatatactgaaggtttgtgttgggtaatgcactactattatgaaggtgtctgctcgtggcaatggtaatttagcttgagttggtcaatataggttgaattgtgtaaaggtttattttcaactctttgtcaatggccttaaattgttatctgtctacctcaccatgcacaggttttacccttaccattatgctccttttgcttcggatttgagtggtctaggtcaactcaatataacttttgaacttgggtcaccattcaaaccgtttgatcaacttatgggagttttcccagctgctaggtgaatacactaagataatgttccctagatcccagttccttaatgttgtggattttttgtaaaggttgtggcattacatttcagctcccatgcactccctgtacagtatcggcaattgatgacagatgcaaattcaccaataattgatttttatccaactggtatgcttatatttgtgatgtttgatatattttttattacctttccactttataattttacactgttttcacccatttgtgatgcagattttgaagtagatatgaatgggaagaggtattcatggcaggtaaattgattataatagggcatatatttatgtaatgtaagtataccttcatgtttaatctatatacttgaatttggcaggggatagcaaaactgcccttcattgatgaagcccggttgctagctgaaattaaaaaagttgagcatactttgacggtatggcaacaaaacagattgtcattcagttgtgcttttataatccatggaataggagatcttgttctaagatatgcatcgcttttcttaccttctgtacagcctgaagaagcaaggagaaacagtatcatgttcaatatgctttttgtgaatggctcccatccactttcgccttatatctactccctcaacagcaaatttggacacctacctgacagagagcgaaatgaaattaaagaaaagattgatccttcttctaggttagctaaccctattaggatccttgatttactgcaaatgctatttttattagtttaaaagcatatggcatttgcactgaccagtgttgtagattctgtaatgccttctgtgtcaggcaaaagttctggctagcataggccactgaattgtttttttcttcttattatttccttaacgcagctgccttgttttgtgcagtggaggaatgaatggttacatctcactttgtagtggggatccgtgccctcctgtcttcaggtctcctgttgatgggttggaggatatcatggacaatcaagtgatgtaagttcaatcccttctgggaaattaagataatatgatttattggcaatctaatttaccatcatgtcatctgtcttaattagatgtacaatctacaagcttccggattctcataagcacatcgctcgtccacctgttggtgtaatcatacccaagaaggtacatctctatcatttttgtctagtatcaacactagtaacatctttccacttgagaaatggtaataacaaataagtaaactctgcagactgttgaagctactgacctgaaaccaccacctgtgctatggcatgaagacagtggtaggaggcctcatgataataacaacaggaggccttatgaaaacagcaacaggtacactatctaccttttgtgttattttgtcaatccttgcaacgtcattgaaaatcctaaccttctggtgaaattgtttaggcaaaacccagctggagcaatatctggccgccaacttggggaagctgcccaccgacttgtggtgaacagcttgaatgctcggagtggcggacaatacaataccccatcgatgccatatcaaaccataatgaatggcatgccttatccaaatgggattccaccaaggatggagcagcctgctcctggctggcatgttcctggtgatctaccaaatggtcaggtaccaccagcttatgcatcatcatcaggtcattatcagaacgataggtctgggccttctcagtatggacgagataaccatggaaggtacccatatgcaagagacaaccaccatgattcaagaggaagggttccaccataccatcagagcggtggaaactcgtatcaatcacattctgctccatcagcaggtcctggacggtatgcgcaacccccaccatatgctggggggtatggtaggagctaccagcctgcaccgtatggaggtggtcagcagtggcagcagcagcagcaacaaccttatggctcctatgctgggtcagggccttatgggggtggggcacctcctgcaaggcctaattcacgaccacagcagtcgcagaaccgttacaataccttagataggaattctaacaggagacctccaccaggtcatgggcgacactaagcccaacctctgtttgttgtacctgtggttttgttgttagaaaacctgcgtactaaaatagagaacctcagcctagtaaaacagagaacctcagcttagagatatatatagagcaatttgcatagtctcgtgatcgacaagctgttatagcatcaaaggttctaagaggcatgtgttgtacaatagaattgtcagttcatcagatactggtggctgactgccgtttttcatttgtggcactcattattttagccccctaaggttgggaatatgtttaacaagttttgtatttttaagataataatgttctaggttaaatttcagttttggtggatatatgtcaaaatcttgtattcttttcttatttataatggaattgtggttttccctctaacctgtcgtggttgtcaattgagaacaagtggttattttttgctggcttataaaatttttctgttaatat

SEQ ID NO: 9 - protein, 1068 aa, Oryza sativa indica
MGVPAFYRWLAEKYPMVVVDVVEEEAVEIEGVKVPVDTSKPNPNGLEFDNLYLDMNGIIHPCFHPEDRPSPTTFAEVFQCMFDYIDRLFVMVRPRKLMYMAIDGVAPRAKMNQQRSRRFRAAKDAADAAAEEERLREEFEREGRKLPPKQQSQTCDSNVITPGTEFMAVLSIALQYYIHLRLNYDPGWKQVKVILSDANVPGEGEHKIMSYIRGQRNLPGFNPNTRHCLYGLDADLIMLALATHEVHFSILREVVYTPGQQDKCFLCGQVGHLAANCEGKVKRKAGEFDEKGEAIVPKKPYQFLNIWTLREYLEYEFRMQNPPFPIDFERIVDDFIFMCFFVGNDFLPHMPTLEIREGAINLLMAVYKKEFPSMGGYLTDACTPDLNKVEHFIQAVGSYEDKIFQKRARLHQRQAERIKREKAQAKRGDDLDPHVRDDLIVPVARFQGSRLASGPAPSPYEQNGSDKNNGGKNSRARKAARVSSSGSSIAAAIVEAENDLEAQERENKEDLKTMLKDALREKSDVFNSENPEEDKIKLGEPGWRERYYEEKFGARTPGQIEEIRRDVVLKYTEGLCWVMHYYYEGVCSWQWFYPYHYAPFASDLSGLGQLNITFELGSPFKPFDQLMGVFPAASSHALPVQYRQLMTDANSPIIDFYPTDFEVDMNGKRYSWQGIAKLPFIDEARLLAEIKKVEHTLTPEEARRNSIMFNMLFVNGSHPLSPYIYSLNSKFGHLPDRERNEIKEKIDPSSSGGMNGYISLCSGDPCPPVFRSPVDGLEDIMDNQVICTIYKLPDSHKHIARPPVGVIIPKKTVEATDLKPPPVLWHEDSGRRPHDNNNRRPYENSNRQNPAGAISGRQLGEAAHRLVVNSLNARSGGQYNTPSMPYQTIMNGMPYPNGIPPRMEQPAPGWHVPGDLPNGQVPPAYASSSGHYQKDRSGPSQYGRDNHGRYPYARDNHHDSRGRVPPYHQSGGNSYQSHSAPSAGPGQYAQPPPYAGGYGRSYQPAPYGGGQQWQQQQQQPYGSYAGSGPYGGGAPPARPNSRPQQSQNRYNTLDRNSNRRPPPGHGRH

SEQ ID NO: 10 - DNA, 7239 bp, Oryza sativa indica
atgggtgtgccggcgttctaccggtggctggcggagaagtacccgatggtggtggtggacgtggtggaggaggaggcggtggagatcgagggcgtcaaggtgcccgtcgacacctccaagcccaaccccaacggcctcgagttcgacaacctctacctcgacatgaacgggatcattcacccctgcttccaccccgaggatagggtacgtcgatgttttttttttgggcgtttctgtttgcgttgctctggggtttgtttgggtgttcgtatggtgtccctccgctgtacaggtgatggatttgtgtatgcctgattgatgtacgacggagctgtggcagaggagatagtttggtgaaatgattttgtagcttcctgttttgtaatttacctgagcgttaaacataattttgcaaataaccacaaactgctaaattgccacatgagccatcttaggatgaggatgctgctgtagtttaatcttcaaactctgcatcgcaaatttgttaatttgcctttttgagttatgtttattttcattcggtggacgcaccagtttagactgcatgatgctactctgtcattgccctgcatttctcttcgaacaaatattttaatgtttactgattgctcaagtgctttctgatttctctgctgaccatgatttcttgctattgtgttatcttttacagccctctccgacaacattcgctgaggttttccagtgcatgtttgattatatcgataggctatttgtcatggttcgccctcggaagcttatgtatatggccattggtacgtataacatctgatgtgtaggaattctcttactacctttttccttcctaaaggattaggagtgtttagtgtcattgctcttttacctgttggtcatcatcatagaacagcttgccatactgccaactaatgcctttgttgaagttagctatatactatgacgagtcatttacatgagttttgaatcaggcataatcctcacccgtaaactatttatttggcagcatagtacctgtgcttgtaaacttttgctgtccagggtcctgttttttaccacatatgttttgttcttgtcgatggtttcttgttgatattttgtattatcttacaatggttttacagatggtgttgctcctcgtgccaagatgaaccagcaacgctctagacgattcagagctgcaaaagatgctgctgatgcggtcagtattctagtgaaattatcttcctttatgcagtgtatgataatatgtcaacatttaaattcccattcatcgacggcttcaaggggtagaattgcatttatggcttattatttttctatctccaggccgcagaagaagagaggttgcgtgaagagtttgagagggaaggcagaaaacttccaccaaaacagcaatctcaaacatgtgattctaacgttattactccaggaacagagtttatggctgttttatcgattgctttgcagtactacatccatctcaggttgaattatgatcctggatggaaacaagttaaagtaagcatggttttaagcctatcagttttgttttggcattacattctgtcagtatgttccatatgttaacttcacaaactctaaggtttacattgattggaaaggcaagtatgaaaaagtcatgtgtattctggttaaattcttttgaaggttattctttctgatgccaatgttcctggtgaaggggaacataaaattatgtcctacattcgcggtcaacggaatctccctggatttaacccaaatactcgccattgtttgtatggcctggtatgttctataaacctattttttatatattttaattgggcaatgtttcttctgttggtcctgctaatgcatttttttatatatactcttgaatgcaggatgcagacctgatcatgttagctcttgcaacacacgaagtacatttctctatactgagagaggttagctctcctcaggacctagaatcaatgcctttttgtgtaaatacattttaccatgttatgctaactaaaagagcggattagttccttatgctttcttgtttatgttatttgttttctctctcttctgactatattttggtttcttgcaggtagtttatacacctggacagcaggacaaatgcttcttgtgtggtcaggttggacatctagcagcaaactgtgaaggaaaggttaagagaaaagctggggagtttgatgaaaaaggtgaagctattgttccaaagaaaccttaccaggtaatattgtctctttcctttgttgcaatttgtggtctaataagtacactttatgaatttgctgaggaccttgttttattttaattgctccttgtgtgacattgcagttcttgaatatatggaccctccgggagtacttagaatatgagttcagaatgcagaatccacctttcccgattgactttgaacgcatcgttgatgatttcatcttcatgtgtttttttgttggcaatgattttcttccacatatgccaacattagagattcgtgaggtttgttacctgttgtaaagctaaatgttttactggatatcttcgtttgaagctagtagctatgaaattatcagattactacttactagtaaattggatatttattttattcctgcttacagttctcttgtagatttgttattgcatgatgttaacttggcttttctgcagggagctattaacttgctaatggctgtatacaagaaggaatttccttccatgggtggctatctaactgatgcttgcacggtaattaaaaagccactcctattgaagttacccatttgtgtgtgtgtgtgtgtgtgtgtgtgtgtttgtttgcagaaagtagtattttattccagctgatccaagttggtatttcagtattaacatgtttttcatctgttacattagccggatctgaataaagttgaacactttattcaagcagttggctcctacgaggataaaatattccagaaaagagctcgtttgcatcaggtgattttattttattttactccttagccttttcaattcccttgttatcatgttatcatctggcacctaatctttatttttactccatgccactatgcgccaaggggttcaccattagctatcagttgaaatttctgtttgtatttggcaatgtaatttccttatttgcaaagttcattgagtgtttattttgatatactggtaatttcagcctgatgtactgcgttatatcttctaggacatgttgtctataaacttccttcaaattggaggtagtgattgtgaatttgccacctgccaccaatagtccaacacactgcatttcttactaattgctccgtgtattgttttaatattacaccgtatatcttctctatgctgctttcttcttagctagccttgtaaattcacaatataacatggaaaggttcacaatgaccagataatccttgacatgtaggttagtgttttttttttttacaatacctctaaagaagttaattctacaattagcatgtgtgaacactgccaatccttgaaaagaagtaaatgctatgctccgtagaaatcacattatgttttgaacaatattgagacattttcgattcccatttagctagtaaaatcagaaaaaccagttgattgttcaagtatcttctaaactagttatttgttcaaggtttcctcccccatactttcccctagaatttaaatgttacccatggaaaacttctgcatgcagcgtaccacgtctgtatggcttaggcatgttccatcattatttatccttagttatgcaaagccttctgatatgatcaacatgaactcctgagaaataaatgtttgcaacactattctatatatctggcggtcaaatattcaacaagtcattgatgttaagttcatatccttgtgtagcgtcaagctgaaaggataaaacgagaaaaggcccaagcaaaacgaggggatgacctggatccccatgtcagggatgatcttattgtacctgttgcacgcttccaagggtctcgccttgcatctggacctgcgccttcaccatatgaacaaaatggatctgacaaaaacaatgggggaaaaaatagtcgtgctcggaaagctgcacgggtatcatcgtcaggttcaagcattgcggctgctattgttgaagctgaaaatgaccttgaagcacaggtagctcatttcatctggctccaggagaatttatatttgtttttgatgaaacatttttgtgattttatctgacacttgttcctgtatctttcttaggaacgcgaaaacaaggaagatttgaagacaatgcttaaagatgcacttcgtgagaaatctgatgtttttaattctgaaaatccagaggaggacaaggttagtgtttacacacaagtcatgccactgctgtgctggaccttattatacgtttaattaaagtgtttactttccttttattttaactcatttactttctattagctatttgtttaaatgttttcaatttagtcataacattgtttgtcttctcagataaaacttggagaaccaggatggagggaaaggtactatgaagagaagtttggagcaagaacacccgggcagattgaagaaatacgcagagatgtcgtaagtgtcttgaatacctttgaattactgcattgcctgataatggtgtcatgatgatctattgctgttaatatatagttgtacgaacttccataaaagatgccatttcatcgctatttgagcatgtactcatttattatcttatatgtatgatgtgccctattcatcttcttcaggttctgaaatatactgaaggtttgtgttgggtaatgcactactattatgaaggtgtctgctcgtggcaatggtaatttagcttgagttggtcaatataggttgaattgtgtaaaggtttattttcaactctttgtcaatggccttaaattgttatctgtctacctcaccatgcacaggttttacccttaccattatgctccttttgcttcggatttgagtggtctaggtcaactcaatataacttttgaacttgggtcaccattcaaaccgtttgatcaacttatgggagttttcccagctgcgaggtgaatacactaagataatgttccctagatcccagttccttaatgttgtggattttttgtaaaggttgtggcattacatttcagctcccatgcactccctgtacagtatcggcaattgatgacagatgcaaattcaccaataattgatttttatccaactggtatgcttatatttgtgatgtttgatatattttttattacctttccactttataattttacactgttttcacccatttgtgatgcagattttgaagtagatatgaatgggaagaggtattcatggcaggtaaattgattataatagggcatatatttatgtaatgtaagtataccttcatgtttaatctatatatttgaatttggcaggggatagcaaaactgcccttcattgatgaagcccggttgctagctgaaattaaaaaagttgagcatactttgacggtatggcaacaaaacagattgtcattcagttgtgcttttataatccatggaataggagatcttgttctaagatatgcatcgcttttcttaccttctgtacagcctgaagaagcaaggagaaacagtatcatgttcaatatgctttttgtgaatggctcccatccactttcgccttatatctactccctcaacagcaaatttggacacctacctgacagagagcgaaatgaaattaaagaaaagattgatccttcttctaggttagctaaccctattaggatccttgatttactgcaaatgctatttttattagtttaaaagcatatggtatttgcactgaccagtgttgtagattctgtaatgccttctgtgtcaggcaaaagttctggctagcataggccactgaattgtttttttcttattatttccttaacgcagctgccttgttttgtgcagtggaggaatgaatggttacatctcactttgtagtggggatccgtgccctcctgtcttcaggtctcctgttgatgggttggaggatatcatggacaatcaagtgatgtaagttcaatcccttctgggaaattaagataatatgatttattggcaatctaatttaccatcatgtcatctgtcttaattagatgtacaatctacaagcttccggattctcataagcacatcgctcgtccacctgttggtgtaatcatacccaagaaggtacatcatttttgtctagtatgaacactagtaacatctttccacttgagaaatggtaataacaaataagtaaactctgcagactgttgaagctactgacctgaaaccaccacctgtgctatggcatgaagacagtggtaggaggcctcatgataataacaacaggaggccttatgaaaacagcaacaggtacactatctaccttttgtgttattttgtcaatccttgcaacgtcattgaaaatcctaaccttctggtgaaattgtttaggcaaaacccagctggagcaatatctggccgccaacttggggaagctgcccaccgacttgtggtgaacagcttgaatgctcggagtggcggacaatacaataccccatcaatgccatatcaaaccataatgaatggcatgccttatccaaatgggattccaccaaggatggagcagcctgctcctggctggcatgttcctggtgatctaccaaatggtcaggtaccaccagcttatgcatcatcatcaggtcattatcagaaagataggtctgggccttctcagtatggacgagataaccatggaaggtacccatatgcaagagacaaccaccatgattcaagaggaagggttccaccataccatcagagcggtggaaactcgtatcaatcacattctgctccatcagcaggtcctggacagtatgcgcaacccccaccatatgctggggggtatggtaggagctaccagcctgcaccgtatggaggtggtcagcagtggcagcagcagcagcaacaaccttatggctcctatgctgggtcagggccttatgggggtggggcacctcctgcaaggcctaattcacgaccacagcagtcgcagaaccgttacaataccttagataggaattctaacaggagacctccaccaggtcatgggcgacactaa

SEQ ID NO: 11 - protein, 965 aa, Populus balsamifera subsp. trichocarpa
MGVPAFYRLLADRYPLSISDVIEEEPQEDSNGNSKPIDVSKPNPNGIEFDNLYLDMNGIIHPCFHPEGKPAPATYDDVFKSIFAYIDHLFALVRPRKLLFMAIDGVAPRAKMNQQRSRRFRAAKDAAQAEAEEERLRKEFEAEGELLSVKEKPETFDSNVITPGTQFMAALSTALQYYIQSRLNHNLGWQNTKVILSDSNVPGEGEHKIMSYIRLQRNLSGFNPNTRHCLYSLDADLIMLSLATREVHFSILREIVTLPGQQDKCFLCGQAGHLAAECRGKQGDDALDWHVVDDTPIHKKKYQFLNIWVLREYLQYDLDILNPPFAIDFERIVDDFVFLCFFVGNDFLPHMPTLEIREGAISLLMHIYRREFSAMGGYLTLAGEVFLDKVEHFIQCVGVYEEQIFQKRTRIQQAFDNNEEMKLKARRESSEVIQAPVVDKVKLGEPGYKERYYAEKFELSNQEEIDKVRKEVVLKYVEGLCWVCHYYFQGVCSWQWFYPFHYAPFASDLKDLGEVELNFFIGEPFKPFDQLMGTLPAASSNALPKEYRKLMTNPSSPIHRFFPSDFEIDMNGKRFAWQGIAKLPFIDERKLLAQTKKLESTLTEEEQIRNRVMLDLLYIHPVHPLAQLVISYYQQNDHLSEGERFAWEIDTRASGGMNGCLWLYERNVRRSVVPSPILGLPALEGNQVLNITFLNPKNRAHIPEIPEGVVMPEKIVKPVDLKPFPTLWHEDNGRRQQGRERPQVQRAIAGPFLGDAAHRLVKNTLNIKPNGSSSRVFDQQLYHNIPGNYTFYRPRPAGPAGYGRGYWDDPNYHYAQHSNQQGLMSNPRYRSLSNGVQSNRHNFRTQDGVQYHQQYHNLSTGVSALTVEENIRSRAPAVISPRMPNPGNTPNLQNQAEQNTGLLSSPPTNWINKTAAGNTGMYFKQKSTSIGPNEKQVKQVYQVKTQVAQETPDIQAQETPDLKQQ

SEQ ID NO: 12 - DNA, 10955 bp, Populus balsamifera subsp. trichocarpa
atgggtgtaccggcgttctacaggcttctcgcagaccgatacccgctatcaatttcggatgtgatcgaagaggaacctcaagaggattcgaatggaaactccaaaccaatcgatgtttcaaagcctaaccctaacggtattgagtttgacaatctgtatttggatatgaacgggattatccatccctgcttccatcctgaaggcaaggtacagtttaccaacttatacttgtatagttttctggaatcttagcttcttagggtttaaaaggaggttttttgtatttgtaattgaattgctgttaaattttgatgcaaatgcaattgactgtcgttaaattttgatggtttttgctcgctgcaaagctatacgtatacagattgtaaagttttagctagggtttaaaagaggggtttttttgtgtgttgttggatgtagttgttgattttgatggatagtgtttaaaatgaacattcatttataattttacacatgttatagggttttgttaaggtttaaaagattgaaagtttttgtgtgttatttaaatgctgtccatttttcatggttagtattgaatggaagcatatatttacgcaagttgtagaattttatattgtaagatttaaaagaggaaatctttgattttgtttgtgtgaattaattgttgtcaattttgatggtttgggcttaaaagaggtgttttttttttatatatagtttctgtgtgtaattgaataagttgaatggtttcttacttttgattttatttacagcctgcacctgctacttatgatgatgtgttcaagtccatatttgcttacattgatcatttgtttgctttggttcgtccaaggaaactactttttatggcaattggtagggttcttgttttattgaatttgtttttttaatcggttttggtatttgttgaatttataatcatttaaagttcattgcttttttcctgggataattgtagatggggttgctcctagagctaaaatgaaccagcaacggtcgcgacggtttagagctgcaaaagatgctgctcaagcagtaagattgagataatttaatggtttttttagtggatcctcttttgtagaagcgattcattttatttatgatgtatgtaggaagctgaagaagaaagattgaggaaagaatttgaggctgagggggagcttttatccgttaaagaaaagcctgaaacatttgattctaatgttatcactcctggcactcaatttatggctgctctttcgactgcgcttcagtattatatacaaagtagattgaatcacaatctgggctggcagaataccaaggtgtgatggatgttctcctttcacttagtttgttatagatcggatcattccttgtctttggagtaatcccttctgtatcttttgaatatactttcggcttttcaggatactttgtgttctctaaacttatttatggggctgcattgggtgattatttgaaggttattctttctgattcaaatgttcctggtgagggagagcacaaaatcatgtcttatataagattgcaacgcaacctttccggtttcaatccaaacacacgccattgtctgtatagtctggtgagcgtgtttctgatttgaggcatttcagattttattgttttattattgttttggcatctaactgctaatcctatgaaaatattttgtttgtggtcaggatgcagatttgattatgctgtcattagccacgcgtgaggttcacttttccatattaagagaggtttgcagctcatatcctaaagttggcatcatttctccatcgattattggtcagtcataaattttatgtgcgttgattgtacagatagttacacttcccgggcagcaggataaatgttttctctgcggtcaagctggtcatcttgcagctgaatgtcgtggcaagcaaggagatgatgctttagattggcatgtggtagatgataccccaattcacaagaagaaatatcaggttgaagaacagacatcaactgtttccatttgtttccatctcgtcgtagtatttctaagcagtttccttatttgaacagttcctcaacatctgggtgctgcgagaatatttgcaatatgatctggacattctgaacccaccatttgctatcgactttgaacgaatagtggatgactttgtgttcctgtgtttttttgttggcaatgacttccttccgcatatgccaacattggaaattagggaggtatgtaagtagcaagcaatggttatatgaatgagtttgggactgaacaacaggctgagttccatttgtttagtttttcagtgatcaactcttgacaaacaattattatgagatgaggtggtttttgttttgtagcttgtttgtggtgctcagcttctagttcgtttggatgctttttagctaatattaagtgacattgatctgcttttgtcctccaatctctcctctacttcttactatgaaatcttttcttcttttgtcaatgaaaaagaatgatattgatgtttttatttatattggccatagttttgttttgaagttctgttgcttcatggttttcactttgctgttcttcctggtgctatctacactatatattgactcttcagttttatcaacttcatcatcacgtcctttcctgattcagggtgctataagcttactgatgcatatttatagaagggagttctctgcaatgggtggttatcttactcttgcgggtgaggtaatttctcttttttatgttctctacaatacttgtgcctttgactatactctggaattaatttatgtttctcattatatgaaatctgttaaagatttggaatacgacattagtctaattttatatcatgcatccatttttttcatttatcacaattatgatcaggtggaaactgtgcgcaacacttgaaaatttagattaacattgttaatgatggtctgttcattcttgttgttttcctcccacctttgctctaactgttcaaaaccaagttttttttctcattttagtttagataacttctccttttaatcaagtggatttattcaataaaatctgctaataaaaattaataaataatttacaaagttatagacaagaacttctgttagagtttgtagtgagaggtttttgttgtttatgagcaatgatgttttaagacttctggtgagtgtagtgtttctaaaattactcagattcatctaagttttctaactttggacttgatttcgaactcatttagcccttccatttcatcctatagccatctgaattaatgtggaccacttgagacaagaggatactagcgataacatgggatttttgctttatgtttgctggaagatatcattgtaaatattagaaagttcatacattatcaatgcatttggttgtctccaaatagaattagatcttagctgcccaattccaccctttcctctgtatctcttttatcacacttatttgtttggaaattgaattcaggcttggctcattttatatgcttgaatttctagaatgacttgcttgctaaacatgtgctataatattcagtgggttctagccaagatatagacatgttatagcctgtcttgacccctagttattctaaaatactttttttatttctccttttctctcaaagaagttcctttttggctctgaaagtcttacttggttagataatctttttattagttattgctctttgtcacccatatcatgaaatagttacatctatctgctggatttgccaaataaacttgttcttcagtgcttctttccccctttaaatcaccatggcttgtatttcatactttttatgctgctttgttggttactttcagggaatgattttgctccactattgcgagcatgagttctaatgaatgttctgatttcaattagcttctgtatacaggttttcctagataaagtagaacactttattcagtgtgttggtgtttacgaggagcaaatctttcagaagaggactcgtatacaacaggtgctttctttttccatgcatggacttatagtcttcagttctcatccttgggtctaatgcaaacatctgcatttatttttactatttattgctggtaatagcatgttcacttgttggctagaaactttttatgtttagatgacaatttatgtaaaatcttggcaatgatttaacttatctgtaactgtaaatttgctagatacagcaggtaggatgttggccctaaatgaaaccccatatgctgctcctgatttactttattagtagaagtattaggttctgtaaaccattacaaaatttaagaactccgaaaattggtctagtgataagatttttgctttgtgtgctagagctgaagaaatttaggggattcccttatcttgtttggaatgcttagttctctaagatttacgtcaaatttttatacacacattgcaaagcagactgtattcttctttctatttttcatctgtggcaaattttgagttttggagtcatatacatactgtcttctctggttatctttttgtttgtacttgtgtggataatccgttttcaggctttcgataataatgaagagatgaaactcaaagcaagaagagagtcctctgaagtgatacaagcaccagttgtagataaggtttgagcctataatattttcatttgatgatatggatttaaataaatgtgttgtgttctgaatttgcctgcgaaaaattgtgtaggatctatattaaacaaaaatgaaccaattatcaattccttgccagttagtttttgacagaaacctcaatcctctctcatttttttggacttccttctccatgtatagacgaaagctcgttagaggagttgatgcttaatattgtttctaatttctcaatacaggatgttggtcataactcttcgagagaaaggaatataaagctaattttgtatgattcccatgttgtgtttggtgacctaaggatttttcttttcttaatagggtttttatgatttaaaaaataataattgcttttgctttctaactggtaaatctgtaacttacggttgtgccttaagttccttgtcatcagaggtagattttattttgtagataaacagcgaagattattttgattcagtagtgtcttaatattgaacatccatttactctagcaggctcgttttctgtttgatatttgcaagtttgcttatccatggccttgcatcttgtgggccaactgaggacaatgtcagcccatttgaaggctagtctggttgtccatttttcctggtataaaatttggttcatttttcttcttgtcaggttaaattaggggaacctggatacaaagagagatactatgctgagaaatttgagttgtcaaatcaagaggagattgacaaagttagaaaggaagtggtaagttgttttttcctaatcatgcaattagcaccttagcatatccatatagattattatctgcattttctcttggtagcaaaattttgtgtgtacacagtagttcatgacaaccgtctgtctcccccaaccagtccttcctctccttaagaaccccaaggccccaacatcaaagctgcaaggttgcttgtacatattttttttcctgtagttatatggtcttcattctgagatcccttggcatgatgaagtacattaaatgagtctttcaatcaccctttttaattgatacaccatggggtggttttcgttatagttattagtttatctgggtgaccttgagtgctacttgtctcactgttgtgctatcaacttgctgttggcttctcatttccagttcctaagagttcatttattagtttatctggatgatctttgggtgtcattagtcttattgctgtgctatcagcatgctattgcttcttgtttccagcctcaaagagttcacatttttttcaacaaatgaatatgttcttcattgtgattgtatttgttttattatgatctcaagttccattattttctccaacttttcattttgagatttcttaatttatattgactttagtttgaaggtgtaatataataaggtgaacttcacagatttacatgaagaaaataaggctgcttttgaaaaatcaaggagttttgcatgtttttccttgaatgggaacactattatgtactatcttaaccaaaggaaaaaaaaagagaataatgaatagatagagaaaaagtcagttgcttgtcatcatataacaaatagaaatctggtgtgacagaagatttggatgtaggaagtatgccggatatggtttctggagtataatttttttttatgttgctaatgattcctttttggtctcccctgtccctgtcacttgttttctgtacaaccaatcatgtatctgaagaaaaaagaaaataacatttgatgttgttcatgcaggtattgaagtatgtggaaggtttgtgttgggtctgtcattattacttccaaggtgtttgctcttggcaatggtcagttcaataaaggcagaaactagaaagtcattatacttcttttcactggctatttaaatttttttattggagtttcaagcacaagcatgtgcccaaaaatacaaacctatatatgaatgcataatatcttgaagctctatacatgagtgaagaaataatggaaaagaggacagtaagttcattgaaagttgagaactatagttctatccgtttcgaaggtggttttgaccctgagctcttaagcatatgacctgccaaacaatcaaatttatatggcttgtgacacctaaatttaactttgccatcactctgctcatcaaatggacaaaaataggcttccattcactctataagcttatatgatacggttccttactgcaattagttgaagttggaattctctgttgtctgtttcaggttttatccgttccattatgctccatttgcttcagatcttaaggatctgggtgaggtggaattaaacttttttattggagagccattcaaaccctttgatcaacttatgggaaccctaccagctgcaaggttttttttctttcttttttatgtgagctccttgcattttcaattttactttggatactctgtctgattattctttctgtgctgtgtgtcatcctttgggcatactacagttctaatgctttgccaaaagaatacaggaaattgatgaccaatccatcttcaccaattcatagattctttccctcaggtaaaagaattgattgaaggatattgattaatttacctcaaggaagtgtttatcttcttcacaagattaatggggtttctttattgtggacagattttgaaattgacatgaatggcaagcgctttgcttggcaggtaaattattctgtattggttatcaatacatgtgttttaactataccttttctttttcaactgattatatttaccttatatttattggaagctagggtattgccaaattgccattcattgatgagaggaaattgcttgcccagacaaagaagcttgaaagcactttaacggtatttaggttttttaaagtagtatccaataatttcatccatcgtaaactatattttaatttctcttgtatgtgttggccattttattagaggctttcactctgcacatgttcttgtaaatataaactgctacctgctccttgctctcttggatgttgagctattcttttggaggcttttactctgatattttcagagatttgaaaaagaaaagtatgcaattaatatttgggaaaacatatttaatggagaaatgccactgtctgagagatcaattttgtgcaaagagaaggattcgtgatgtgatattttaaaaaacctgttgagattcctagttataatgcatttctactgatttagattctaggaaacaatatcaattcaaatactgatttcggagaaaaaatatttatttgttacaaatgcttttgtcaccaaaggttcctaaagtttatttcttatgttactgtaatggaggttgggctgttaacataaactcgtattcatttatcttattacttgtgtaactttttaatagtcaattatttgattgtgtgatctgatagttttttgctaagttttgtatttgaataaatccatgttgacttttgagtgatttccataccagatggaaaagttttttagtttctgatatatcctcctgtacttaaatttctcctgaaatttaatttgtaagcaccaagggtagtttagagttggcagtcagttgtgttcacacccctataatgggtttgatccttcagaagctaaaccttgctgttttgtggcatatcagtcttctttttagtttggatgtcaagtatgttaatctttgatttgctatttgcaggaggaagagcagatcaggaatagagtgatgcttgatttgctatatattcatcctgttcatccattagctcaactagttatttcatactatcaacagaacgatcatttatctgaaggtgaaagatttgcttgggaaattgacacaagagccaggttagttggtcttagtgttgattgatcttttagtttgaagttgtcaaatcattatttatgtgtgctggctttattatctcattttacactaaccttttacagtggggggatgaatggttgtctttggttatatgagagaaatgtgcggagaagtgtggtcccctctcctatacttggattgccagccttagagggtaaccaagttttgtaagttccaactttttccagtgctgttttcataagctgattgatccttggtgtaatgttgtgtactttttctatgcagaaatatcacatttctaaatcctaaaaaccgtgcacacatcccagaaattcctgagggtgtagtcatgcctgaaaaggtgtgtgagctctagatgcctgtaaatttggtttattttgttatacgcagaaaagcaaggacatgaaatttctgtcaactaatgatgtggcagtgattcaattcatgcataaaataatgtaatagtgttattgggtttcatagtttagagtttactggagcagtaaactgggattcttctgtgaaaattaattggccatgcctgaaaaggtatgtgagctctagatgcatgtttttcgaaggtattgttgggcagtaaactggaggcagtccttgtgttcaaggaaattgagtttggtcatggtttctgtttctggaagttcccccctgcatatgggtggaagtgctgatgcaattgtaatataatggttatgttaagtttgcaggtgcaaggattctcatatgttgtttccttgaaggcattttctttggataatgtgttgtgccgtcacagggtttgatgttagttaatgatttgatttcacaaatgtccatggctaatttgaggcatgttcttgctatttgcagatcgtaaaacctgtagatcttaaaccattccccacattatggcatgaagataatggccggagacagcaaggcagggaaaggtacgaatggatttccacatctttgcaatgattgatttgcattggtttgtaatgtttgggtggttaggttgaatatgggtgacatttgttcatgacctgctattcttgacagacaatgcatctcgctcaatttatatgtggagaaaggaagggtttaattgatatcctcttttttttctctgaaaaacatagattgtagggtttagaacttactgcttcgtcttctcttaaatagcctaattcacatgtgctgtctctgtattacaggccacaagtacaacgagcaatagctggccccttcctgggggatgcagctcatcgtcttgtcaagaacacccttaatatcaaaccaaatggttcatcctctagagtgtttgaccagcaactatatcataatatccccggcaattacacattctacagaccaagaccagctggaccagctggttatggaaggggctattgggatgatccaaactatcattatgcacagcacagtaaccagcaaggcctgatgagtaatcctaggtatcgatctttgtcaaatggtgtgcaaagcaaccgacacaactttaggactcaagacggagtacagtaccatcaacagtatcataacttgagtactggggtgtctgctctgactgtagaagaaaatatcagaagcagggcacctgcagtgatctcacctcgaatgccgaatccaggaaacactccaaacttacaaaatcaggccgagcagaacacaggtctactttcatcaccacccaccaattggatcaacaaaacagctgcaggaaacactggaatgtacttcaaacaaaaatcaacatcgataggtccaaatgagaagcaggtgaagcaggtttatcaggttaagacacaagtagctcaggagacgccagatattcaagctcaggagacgccagatttaaaacagcagtaacattttatatttgaagaatcttgcatgtttttgccacactcacattgcagtggcaatgcatgttcatttgaatctttgcataagcgcaagtttttggcgaatttactcggtttgggcaacagtcacagaaactattcctggatggtatgcagctgcaagagctcgtggatgttaaaagagtataatatatgaacagtggcctttttgagatccacttttgatttggtgcgagttagcatactgcatctcacctcattcatgttgcagctgcttttttccagattttcctatttgtttaccagattgtcgtagaaaactgtatcatacaaatgtttggttgtaatcgggcatctatctcagaaatgttgtcagtatgtaccgtattttggatgatgtaattcttgatgtaattctatgcagtgtcctttttagtgactcggaactttttgcttcaggatttctgtgcaaat

SEQ ID NO: 13 - protein, 1240 aa, Zea mays
DGSPTRPPARTHARGHRPSFPFPRTHAPHLSSLRPYRSNQGGSNQRTPLRRELAEAPPHERESRARKRSGGRAGRSEMGVPAFYRWLADRYPLTVSDAEEEEPVELEPGAFVPVDLRRPNPNGLEFDNLYLDMNGIIHPCFHPEGRPAPTTYDEVFKSIFDYIDHLFGLIRPRKLLYMAIDGVAPRAKMNQQRSRRFRAAKDAADAAAEEERLRKEFEAEGRTLAQKEKSEAIDSNVITPGTEFMFVLSTALQYYIQLRLNHTLGWQSVKIILSDSNVPGEGEHKIMSYIRLQRNLPGFDPNTRHCLYGLDADLIMLALATHEVHFSILREVISMPGQHEKCFLCGQVGHLAAECRGPSQPDNSVELPPIHKKKYQFLNIWVLREYLAKDLEIIDAPFKINFERLIDDFVFMCFFVGNDFLPHMPTLEIREGAINLLMSIYRSEFTSMGGYLTDVGEVILDRVEHFIQSVAVNEEQIFQKRARIQQARENNEEKHKMQRENSEEDQYVDKVKLGEPGYRERYYADKFKEEAESKPIVQVRRDVVQKYVEGLCWVMRYYYQGVCSWQWFYPYHYAPFASDLKGLAELEITFFLGQPFKPFDQLMGTLPAASSNALPKYYGDLMSDPDSPLKSFYPKDFEIDMNGKRFAWQGVAKLPFIDERRLLAETRKLEDTLTEEEKFRNRTMLDIIYVRDTHPLITQIIFLYQNYYHLSRTDPYVIPIEPAASGGMNGFLCLSERNWYSVTVTSPVKGFNGIAQNRVLNAAYLNPQYHKHIPEPPAGVIIPAKILKPSDFKPFPVLWHQDNSRRQVRERPQVSGALSGSLLGEAAHRLVKNSLQIRSGNAAGLLYMPYRGAPYGPGNRPRPAGPLGYERGFVDNPYHAHMSRSVPNPHAQFFGDSQANRQPMRILERPDSRSHDAGIRASMSKLTIQEGPRPHQNNRMQNSGYWPNQPHPTHFTGPPAQRPMQNISFTPQRPFQTGGFPQQRPVNGAPPPLPPSNWIGKQPSGCHMSVSPAKLDPRTAPDRQPKQDNPRSQQDKRQQATKVYRVKTQATNVNGLSEPGKQEEPEGLGCCKSTRQVCAWREYAGSWQQRVSMYPSHNLPFDVELFVVLPGKESAKFVRGFRHSFCLVDRTWYGHSLLVVKLLVGLKLFCAGLLHIQHRPVRRLAQLPCSCSISWKLGADVLVVFKLSSLCLERRKHSEFFIVPLNQVESLTLKRTCSSRARVPLGHGIIYIFFFSGMQT

SEQ ID NO: 14 - DNA, 3754 bp, Zea mays
agacggaagcccaacccgcccgcctgcccgcacacacgcgcgtggccatcgcccctccttccccttcccccgcacacacgcgcctcacctttcgtcccttcgcccctacagatcgaaccaaggaggcagcaaccagcgcacgccactgcggcgctaggagctcgcagaagcgccgccccacgagagggagtccagagcaaggaagagatccggtggcagagcaggtcggtcggagatgggagtaccggcgttctaccggtggctggcggaccgctacccgctgacggtgtctgacgcggaggaggaggagcccgtggagctcgagcccggcgccttcgtccccgttgacctccgccgccccaatcccaacggcctcgagtttgacaacctctacctcgacatgaatggtatcatccacccatgcttccaccccgagggccggccggcgcccaccacctacgacgaggtgttcaagtcgatttttgattacatcgaccacctcttcggcctcatccgccccaggaagcttctctacatggccattgatggtgttgctccaagggcaaaaatgaaccagcagaggtccaggcgattccgggctgctaaggacgcggctgatgcggcagcggaggaagagaggctgaggaaggagtttgaggcagagggtaggactctagcccagaaagagaagtcggaggcaattgactccaatgtcattactccagggacggagttcatgtttgtactatctacggcgcttcagtactacatacagctgaggctgaatcacacgcttggatggcagtctgtaaagataatactgtctgattccaatgtccctggagagggagagcacaaaattatgtcatacatccgcctgcagcgcaatcttcctggctttgatcccaatacacgccattgcttatatggcctggacgctgatttgattatgcttgctctagcgactcacgaggttcacttctcaattttaagagaggtgatctccatgccagggcaacatgaaaaatgttttctttgtggtcaagttgggcatttggctgctgaatgcagaggtcctagtcagcctgataattctgtggagctacctcctattcataaaaagaaataccagtttcttaacatatgggtgttgcgtgaatacctggcaaaggatttggaaattatagatgctcccttcaagataaactttgaacgtcttattgatgattttgtgtttatgtgtttctttgttggcaatgactttcttcctcatatgccaactttggaaattcgtgagggtgccattaatcttctcatgagtatctacaggtcggagttcacatcaatgggtggttacctaactgatgtgggtgaggttatattggaccgagtggaacatttcatccagtctgttgcggtcaatgaagaacagattttccagaaacgggcacgcattcaacaggcacgtgagaataatgaagagaaacataaaatgcagagagagaattctgaggaggatcaatatgtggataaggtgaagttaggagagccaggatacagggagcgttattatgctgataagtttaaagaagaggcagaatcaaaacccattgttcaagttcggagagatgttgtccagaaatacgtggaaggtctttgttgggttatgagatactactatcaaggtgtttgttcatggcagtggttttatccctatcattatgcgcctttcgcatctgatctaaaaggcttggctgaattggaaataaccttttttttgggtcaacctttcaagccatttgatcaactaatgggaaccctaccagctgcaagctccaatgcactgccaaaatactatggggatttgatgagtgatccagattcaccgttgaagtctttctacccaaaagattttgagatagacatgaatgggaaacgttttgcatggcagggtgttgcaaaattgccttttattgatgaaaggcgtctgcttgcggaaacaagaaaacttgaagatactttgacagaagaagagaaattcaggaataggactatgcttgacataatttacgttagagatactcatccattgatcactcagattatcttcctgtatcagaactattaccatctatcaagaacagacccttatgttattcccattgagcctgctgctagtggtggaatgaatggattcctatgtttatctgaaaggaactggtatagtgtcactgtcacttctccagttaaggggttcaatggcattgctcagaacagagttttgaatgctgcctatctcaatcctcagtatcacaaacacatcccagagcctccagcgggggtcatcatacctgcgaagatactgaagcctagtgatttcaaaccctttcctgtgttgtggcatcaagataatagtcgtcgacaagtaagagaaaggcctcaggtttctggagccctgtccggttctctcttaggagaagctgcacaccgactggtgaaaaactccctccagatcagatctggcaatgctgctgggttactttatatgccatacagaggtgcaccctatggccctggaaacagaccaaggcctgctgggccattggggtatgagaggggttttgtggataatccataccatgcacacatgtctagaagtgttccaaaccctcacgctcaattctttggtgattctcaagctaacagacagcccatgcggatactagaacggccagactcccgaagtcatgatgctggtatccgtgcatcaatgtctaaactgacaatccaagaagggccaaggcctcatcaaaataacaggatgcagaattccgggtactggcccaatcaaccacatcctactcactttactggacccccagcccagcggcctatgcagaacatcagttttacacctcagcgacctttccaaaccgggggatttccgcagcagagacctgtaaatggggctccaccaccattacctcccagtaactggattggtaagcaaccaagtggatgccacatgagtgtatcacctgcaaaactcgatccaaggacagctccagacagacagcccaagcaggataacccaagatctcaacaggataagaggcagcaggcgaccaaggtataccgtgtcaaaactcaagctactaatgttaatggtttgtcagagccaggtaaacaggaagaacctgaagggtaactgggttgctgcaaatccacacgccaagtttaatgtgcgtggagagaatacgctggctcttggtgatagcagcagagggtttccatgtatccctcccacaatttacctttcgatgttgaattgtttgttgtattacccggaaaggagtcggctaaattttaggtcaggggatttagacacagcttttgtttagttgataggacatggtatgggcattcactgcttgttgtaaagctgttagttggatagctgaaactgttttgtgctggctgactgctgcatattcaacatagacccgtgcggaggctggctcaactgccctgctcctgctctatctcatggaagttgggggcagatgtgcttgtggtcttcaaactgagcagtttgtgtctctgagaaaggaggaagcattccgaatttttcattgtaccgttgaaccaagttgagtctttaacactgaaaagaacatgttcctctcgagcttaacgagtgccataattaggacatggaataatatatatatttttcttttctgggatgcaaaca

SEQ ID NO: 15 - protein, 16 aa, Arabidopsis thaliana
DGVAPRAKMNQQRSRR

SEQ ID NO: 16 - protein, 9 aa, Arabidopsis thaliana
GLDADLIML

SEQ ID NO: 17 - protein, 5 aa, Zea mays
GDVAP

SEQ ID NO: 18 - DNA, 19 bp, Zea mays
gatggtgttgctccaaggg

(19) United States
(12) Patent Application Publication
(10) Pub. No.: US 2017/0298379 A1
(43) Pub. Date: Oct. 19, 2017

(54) FUNCTIONAL MUTANT ALLELES OF EIN5 FOR YIELD INCREASE

(71) Applicants: VIB VZW, Gent (BE); Universiteit Gent, Gent (BE)

(72) Inventors: Dirk Gustaaf Inze, Moorsel - Aalst (BE); Hilde Nelissen, Gent (BE); Marieke Dubois, Boeschepe (FR)

(21) Appl. No.: 15/515,578

(22) PCT Filed: Oct. 5, 2015
(86) PCT No.: PCT/EP2015/072924

§ 371 Date: Mar. 29, 2017

(30) Foreign Application Priority Data
Oct. 3, 2014 (EP) 14187614.4

(51) Int. Cl. C12N 15/82; C12N 9/22

(52) U.S. Cl. CPC C12N 15/8273; C12N 9/22

(57) ABSTRACT

This disclosure relates to novel mutant alleles for increasing biomass in plants and provides mutant plants comprising alleles and chimeric genes encoding the novel alleles.

### CROSS-REFERENCE TO RELATED APPLICATIONS

This application is a national phase entry under 35 U.S.C. §371 of International Patent Application PCT/EP2015/072924, filed Oct. 5, 2015, designating the United States of America and published in English as International Patent Publication WO 2016/050984 A1 on Apr. 7, 2016, which claims the benefit under Article 8 of the Patent Cooperation Treaty to European Patent Application Serial No. 14187614.4, filed Oct. 3, 2014.

### STATEMENT ACCORDING TO 37 C.F.R. §1.821(c) or (e)—SEQUENCE LISTING SUBMITTED AS ASCII TEXT FILE

Pursuant to 37 C.F.R. §1.821(c) or (e), a file containing an ASCII text version of the Sequence Listing has been submitted concomitant with this application, the contents of which are hereby incorporated by reference.

### TECHNICAL FIELD

This application relates to the field of plant molecular biology. More specifically, this disclosure provides mutant alleles, chimeric genes comprising the mutant alleles, and plants comprising the mutant alleles. Plants comprising the mutant alleles are characterized by having an increase in yield compared to wild-type plants.

### BACKGROUND

Leaf growth is a complex process, integrating genetically programmed processes and environmental signals. These environmental signals can trigger inhibition of leaf growth, which, under drought stress, for example, forms a mechanism to limit water evaporation from the leaf surface. During the past decades, extensive research has been performed on Arabidopsis thaliana to study plant behavior under extreme environmental conditions such as severe drought (Xiong et al., 2002; Verslues et al., 2006; Seki et al., 2007; Schachtman and Goodger, 2008). These studies mainly scored plant survival or used final rosette area as a measure for plant tolerance to drought (Umezawa et al., 2006). However, it became clear that the often rather harsh conditions used in these studies are not very representative of natural conditions, in which the drought stress is generally milder and does not always threaten survival (Skirycz and Inzé, 2010). Moreover, it was shown that tolerance to severe drought stress and the ability of plants to continue to grow under mild stress conditions are very different traits mediated by different molecular processes (Skirycz et al., 2011a; Claeys and Inzé, 2013). Furthermore, recent studies pointed out the importance of evaluating stress responses at organ or tissue level, as the response to osmotic stress is highly dependent on the organ and its developmental stage (Dinneny, 2008; Harb et al., 2010; Skirycz et al., 2010; Baerenfaller et al., 2012). Thus, in order to understand the specific mechanisms involved in growth inhibition under water deprivation, it is important to study growth response specifically in actively growing leaves.

Leaf growth in Arabidopsis is consisting of three major developmental phases (Anastasiou and Lenhard, 2007; Andriankaja et al., 2012; Gonzalez et al., 2012). First, growth of very young leaves emerging from the shoot apical meristem is driven exclusively by cell proliferation. Next, cell division starts to cease at the tip of the leaf and gradually all cells in the leaf exit the cell cycle and start to expand (Donnelly et al., 1999; Andriankaja et al., 2012; Gonzalez et al., 2012). Finally, cell division in the developing leaves has stopped and further leaf growth mainly occurs by cell expansion (Vlieghe et al., 2005). Under mild drought conditions, studied in vitro using osmotic stress as a proxy, both cell proliferation and cell expansion are affected resulting in a final leaf size reduction of about 50% (Skirycz et al., 2010; Skirycz et al., 2011b; Claeys et al., 2012).

When mild stress occurs during the proliferation phase of early leaf development (leaves <0.1 mm² in size), cell cycle progression is affected in a two-step process denominated the “Pause-and-Stop” mechanism, involving crosstalk between the plant hormones ethylene and gibberillic acid (GA) (FIG. 1A) (Skirycz et al., 2011b; Claeys et al., 2012). In the first step of mild stress response, the ethylene precursor ACC, which accumulates specifically in growing leaves, inactivates the Cyclin Dependent Kinase A (CDKA), thereby transiently blocking further cell cycle progression (“Pause”). In parallel, the accumulation of ACC induces the expression of the downstream ETHYLENE RESPONSE FACTOR 6 (ERF6). This transcription factor occupies a central role in this pathway as, on the one hand, it activates the stress tolerance mechanisms, while on the other hand, it further converts the paused cell cycle into a definitive cell cycle exit (“Stop”) (Dubois et al., 2013). Molecularly, the latter occurs through transcriptional activation of the GA2-OX6 gene, which stimulates breakdown of GAs. Decreased GA levels further result in stabilization of DELLA proteins, which push cells into cell expansion and endoreduplication (Claeys et al., 2012; Achard et al., 2008).

It is, however, very unlikely that this rather simple linear pathway on its own is the sole regulator of leaf growth under stress. Considering the complexity of regulation of leaf growth under normal conditions (Gonzalez et al., 2012), and the numerous pathways that are responding to changing environments (Skirycz an Inzé, 2010), one can imagine that the integration of environmental signals into leaf growth adaptation is an extremely complex response involving different molecular processes such as signal detection, post-transcriptional regulation, protein complex formation, etc.

### BRIEF SUMMARY

To identify new genes involved in leaf growth regulation under adverse conditions without restricting our windows toward certain molecular processes or biological functions, a forward genetic screen was performed. As a starting point, the easily screenable severe dwarfed phenotype of an inducible ERF6-overexpression line was used, mutagenized with EMS, and screened for suppressor mutants. In this disclosure, different alleles were identified from EIN. These alleles encode for mutant, functional proteins, which, when present in a homozygous state in a plant, lead to an increased leaf biomass.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D: Principle of the EMS screen and general characteristics of the seven identified mutants. (FIG. 1A) ERF6-centered pathway of growth-inhibition and stress tolerance activation under osmotic stress. Upon activation of ERF6, the GA levels are reduced due to induction of GA2-OX6, resulting in a growth inhibition. (FIG. 1B) As a consequence of the pathway in FIG. 1A, plants overexpressing ERF6 are extremely dwarfed. Upon EMS mutagenesis, the indicated mutant suppresses the ERF6-induced dwarfism. The in vitro growth medium contains Kanamycin to select for seedlings containing the ERF6-GR construct, and Dexamethasone to induce the overexpression of ERF6 and the accompanied dwarfism. (FIG. 1C) Expression analysis of the mutants upon transfer to Dexamethasone for the overexpression of ERF6, the stress-related target gene MYB51 and the growth-related target gene GA2-OX6. Mutants sgi1-sgi4 and sgi7 no longer show induction of GA2-OX6 but still induction of MYB51, while sgi5 and sgi6 mutants do not induce MYB51 but have reduced levels of GA2-OX6. (FIG. 1D) Rosette phenotypes of the seven selected mutants, grown in soil in the absence of Dexamethasone. Pictures are taken 22 days after stratification.

FIG. 2: Structure of the XRN4 protein with location of the identified mutations. Based on structure predicted by PLAZA (Proost et al., 2009) and on the observations of Nagarajan et al. (2013), underlined residues indicate key residues for active site function surrounded by conserved stretches of amino acids. Mutants obtained with the EMS screen are indicated in red, with mutants used in previous studies in green (Potuschak et al., 2006). Small arrows indicate point mutants and large triangles indicate T-DNA insertions. (Sequences appearing in FIG. 2 are portions of SEQ ID NO:1, SEQ ID NO:15, and SEQ ID NO:16.)

FIGS. 3A and 3B: Rosette size of the EMS mutants and the previously described ein5 mutants. (FIG. 3A) Rosette size of the EMS mutants at 22 days after stratification (DAS) upon growth in soil. Values were normalized to the wild-type size. (FIG. 3B) Rosette size of the previously characterized ein5 mutants at 22 days after stratification (DAS) upon growth in soil. Values were normalized to the wild-type size. Error bars represent standard error.

FIGS. 4A-4C: Individual leaf size and cellular measurements of sgi7.1 (FIG. 4A) Picture of a representative sgi7.1 rosette (left) and appropriate WT (ERF6-GR) (right) after 25 days of growth in soil. (FIG. 4B) Size per leaf of the sgi7.1 mutant and wild-type at 22 days after stratification (DAS) upon growth in soil. Error bars represent standard deviation. Four biological repeats were done. (FIG. 4C) Cellular measurements of the abaxial epidermal cell layer of representative third leaves from the plants in (FIG. 4B). Error bars represent standard error.

FIG. 5: Rosette size of sgi7.1 under control and drought stress conditions. Rosette size of the sgi7.1 mutant at 22 days after stratification (DAS) when grown on the Weighing Imaging and Watering Machine under either well-watered conditions or mild drought stress. Values were normalized to the wild-type size.

### DETAILED DESCRIPTION

To facilitate the understanding of this disclosure, a number of terms are defined below. Terms defined herein (unless otherwise specified) have meanings as commonly understood by a person of ordinary skill in the areas relevant to this disclosure. As used in this specification and its appended claims, terms such as “a,” “an,” and “the” are not intended to refer to only a singular entity, but include the general class of which a specific example may be used for illustration, unless the context dictates otherwise. The terminology herein is used to describe specific embodiments of the disclosure, but their usage does not delimit the disclosure, except as outlined in the claims. Unless specifically defined herein, all terms used herein have the same meaning as they would to one skilled in the art of this disclosure. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 100), John Wiley & Sons, New York (2012), for definitions and terms of the art. The definitions provided herein should not be construed to have a scope less than understood by a person of ordinary skill in the art.

This disclosure is derived from the unexpected findings that the homozygous presence of specific EIN5 alleles, encoding mutant, and functional EIN5 proteins in a plant that does not express a wild-type EIN5 protein, leads to an increased yield of at least 20% with respect to the wild-type plant not comprising the specific EIN5 alleles. Specific EIN5 alleles that are functional and encode for mutant EIN5 proteins are selected from the list consisting of: i) a protein having at least one amino acid change in the sequence DGVAPRAKMNQQRSRR (SEQ ID NO:15) present in SEQ ID NO:1 or in a corresponding plant orthologous sequence thereof; ii) a protein having at least one amino sequence change in the sequence GLDADLIML (SEQ ID NO:16) present in SEQ ID NO:1 or in a corresponding plant orthologous sequence thereof; iii) a protein having an amino acid change at position 526 or in a corresponding plant orthologous sequence thereof. Yet another specific EIN5 allele that is functional has its splice acceptor site mutated at the nucleotide position 1603 in SEQ ID NO:2 or in a corresponding plant orthologous sequence thereof.

The term “at least one amino acid change in a sequence” means that at least one of the amino acids present in the sequence can be changed (e.g., by mutation) toward any possible amino acid.

In a specific embodiment, the functional mutant EIN5 allele encodes for a functional protein selected from the list consisting of: i) a protein having an amino acid change of G to E in position 105 of SEQ ID NO:1, or in a corresponding position in a plant orthologous sequence thereof; ii) a protein having an amino acid change of A to V in position 110 of SEQ ID NO:1, or in a corresponding position in a plant orthologous sequence thereof; iii) a protein having an amino acid change of Q to STOP in position 115 of SEQ ID NO:1, or in a corresponding position in a plant orthologous sequence thereof; iv) a protein having an amino acid change of D to N in position 236 of SEQ ID NO:1, or in a corresponding position in a plant orthologous sequence thereof; v) a protein having an amino acid change of Q to STOP in position 526, or in a corresponding position in a plant orthologous sequence thereof or the functional allele of EINS has its splice acceptor site mutated at the nucleotide position 1603 in SEQ ID NO:2 from G to A, or in a corresponding position of a plant orthologous sequence thereof.

The EIN5 gene is sometimes referred to in the art also as the AIN1 or XRN4 gene. Thus, a representative of the plant EINS gene in Arabidopsis thaliana is AT1G54490 (TAIR accession, on the World Wide Web at arabidopsis.org), which genome sequence is depicted in SEQ ID NO:2 and its protein coding sequence is depicted in SEQ ID NO:1.

The term “plant yield” or “yield” as used herein generally refers to a measurable product from a plant, particularly a crop. Yield and yield increase (in comparison to a wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeably. As used herein, the term “improved yield” or the term “increased yield” means any improvement in the yield of any measured plant product, such as biomass (e.g., leaf biomass), grain, fruit, leaf, root, or fiber. In accordance with the disclosure, changes in different phenotypic traits may improve yield. For example, and without limitation, parameters such as floral organ development, root initiation, root biomass, seed number, seed weight, harvest index, leaf formation, phototropism, apical dominance, and fruit development, are suitable measurements of improved yield. Increased yield includes higher fruit yields, higher seed yields, higher fresh matter production, higher leaf biomass and/or higher dry matter production. Any increase in yield is an improved yield in accordance with the disclosure. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans derived from a soy bean plant that does not express wild-type EIN5 protein but comprises and expresses allelic variants of EIN5 in a homozygous state, as specified hereinbefore, as compared with the bu/acre yield from wild-type soybeans cultivated under the same conditions, is an improved yield in accordance with the disclosure. The increased or improved yield can be achieved in the absence or presence of stress conditions. For example, enhanced or increased “yield” refers to one or more yield parameters selected from the group consisting of biomass yield, dry biomass yield, aerial dry biomass yield, underground dry biomass yield (e.g., root biomass), fresh-weight biomass yield, aerial fresh-weight biomass yield (e.g., leaf biomass), underground fresh-weight biomass yield; enhanced yield of harvestable parts, either dry or fresh weight or both, either aerial or underground or both; enhanced yield of crop fruit, either dry or fresh weight or both, either aerial or underground or both; and enhanced yield of seeds, either dry or fresh weight or both, either aerial or underground or both. “Crop yield” is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. The yield of a plant can depend on the specific plant/crop of interest as well as its intended application (such as food production, feed production, processed food production, biofuel, biogas or alcohol production, or the like) of interest in each particular case. Thus, in one embodiment, yield can be calculated as harvest index (expressed as a ratio of the weight of the respective harvestable parts divided by the total biomass), harvestable parts weight per area (acre, square meter, or the like); and the like. The harvest index is the ratio of yield biomass to the total cumulative biomass at harvest. Harvest index is relatively stable under many environmental conditions, and so a robust correlation between plant size and grain yield is possible. Measurements of plant size in early development, under standardized conditions in a growth chamber or greenhouse, are standard practices to measure potential yield advantages conferred by the presence of the EIN5 alleles of the disclosure in a homozygous state in a background with no wild-type EIN5 expression.

There are two major options to produce a plant expressing the mutant, functional EIN5-alleles of the disclosure: i) introducing (e.g., by breeding or by genetic transformation) the mutant, functional EIN5 alleles in a plant that does not express wild-type alleles of EIN5 and ii) modifying the wild-type EIN5 alleles in a plant by introducing targeted mutations in the plant so that the mutant, functional EIN5 alleles are present in a homozygous state. The various steps to generate plants expressing a functional, mutant EIN5 allele of the disclosure are discussed hereinbelow.

Methods for Generating a Plant with Knock-Out Alleles of EIN5

As used herein a “an EIN5 knock-out allele” is an EIN5 allele that is mutated as compared to the wild-type EIN5 allele (i.e., it is a non-functional EIN5 mutant allele) and that encodes a non-functional EIN5 protein or results in a significantly reduced amount of EIN5 protein (by introducing, for example, a mutation in a regulatory region such as a promoter), or that encodes a protein with significantly reduced activity. The “EIN5 knock-out allele” may encode no EIN5 protein, or may encode a non-functional EIN5 protein or an EIN5 protein with significantly reduced function. Such an EIN5 allele may also be referred to as an inactivated EIN5 allele. A wild-type EIN5 allele is the form of the EIN5 allele as it occurs in nature.

In a specific embodiment, the activity of an EIN5 protein may be reduced or eliminated by disrupting the genes encoding EIN5. The disruption inhibits expression or activity of EIN5 protein compared to a corresponding control plant cell lacking the disruption. A specific embodiment, the disruption step comprises insertion of one or more transposons, where the one or more transposons are inserted into the endogenous EIN5 genes. The disruption can be a homozygous disruption in the EIN5 gene. Alternatively, the disruption is a heterozygous disruption in the EIN5 gene. In certain embodiments, when more than one EIN5 gene is involved, there is more than one disruption, which can include homozygous disruptions, heterozygous disruptions or a combination of homozygous disruptions and heterozygous disruptions. Methods for the transposon tagging of specific genes in plants are well known in the art. See, for example, Meissner et al. (2000) Plant J. 22:265-21. In addition, the TUSC process for selecting Mu insertions in selected genes has been described in U.S. Pat. No. 5,962,764, which is herein incorporated by reference.

In a specific embodiment, a gene encoding for a zinc finger protein that binds to a gene encoding an EIN5 polypeptide is introduced into a plant or plant cell, resulting in a reduced expression of the EIN5 gene. In particular embodiments, the zinc finger protein binds to a regulatory region of an EIN5 gene. In other embodiments, the zinc finger protein binds to a messenger RNA encoding an EIN5 polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. 2003/0037355, each of which is herein incorporated by reference.

In another specific embodiment, a gene encoding a TALE protein that binds to a gene encoding an EIN5 polypeptide, is introduced into a plant or plant cell, which results in a reduced expression of the EIN5 gene. In particular embodiments, the TALE protein binds to a regulatory region of an EIN5 gene. In other embodiments, the TALE protein binds to a messenger RNA encoding an EIN5 polypeptide and prevents its translation. Methods of selecting sites for targeting by zinc finger proteins have been described in, e.g., M. J. Moscou and A. J. Bogdanove (2009) (A simple cipher governs DNA recognition by TAL effectors, Science 326:1501) and R. Morbitzer, P. Romer, J. Boch, and T. Lahaye (2010) (Regulation of selected genome loci using de novo-engineered transcription activator-like effector (TALE)-type transcription factors, Proc. Natl. Acad. Sci. USA 107:21617-21622).

Additional methods for decreasing or eliminating the expression of endogenous genes in plants are also known in the art and can be similarly applied to this disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods, see Ohshima et al. (1998) Virology 243:472-481; Okubara et al. (1994) Genetics 137:867-874; and Quesada et al. (2000) Genetics 154:421-436, each of which is herein incorporated by reference. In addition, a fast and automatable method for screening for chemically induced mutations, TILLING (Targeting Induced Local Lesions in Genomes), using denaturing HPLC or selective endonuclease digestion of selected PCR products is also applicable to this disclosure. See, McCallum et al. (2000) Nat. Biotechnol. 18:455-457, herein incorporated by reference. Mutations that impact gene expression or that interfere with the function of the encoded protein are well known in the art. Insertional mutations in gene exons usually result in null-mutants. Mutations in conserved residues are particularly effective in inhibiting the activity of the encoded protein. Conserved residues of plant EIN5 polypeptides suitable for mutagenesis with the goal to eliminate EIN5 activity have been described. Such mutants can be isolated according to well-known procedures, and mutations in different EIN5 loci can be stacked by genetic crossing. See, for example, Gruis et al. (2002) Plant Cell 14:2863-2882.

### Methods for Producing a Plant Having Knock-Out Alleles of EIN5 Further Comprising Specific EIN5 Alleles of the Disclosure

In a specific embodiment, a plant in which no EIN5 expression occurs (i.e., a plant having knock-out alleles of EIN5) is transformed with a chimeric gene comprising a plant-expressible promoter operably linked to an EIN5 allele of the disclosure and a suitable plant terminator sequence.

A chimeric gene, as used herein, refers to a gene that is made up of heterologous elements that are operably linked to enable expression of the gene, whereby that combination is not normally found in nature. As such, the term “heterologous” refers to the relationship between two or more nucleic acid or protein sequences that are derived from different sources. For example, a promoter is heterologous with respect to an operably linked nucleic acid sequence, such as a coding sequence, if such a combination is not normally found in nature. In addition, a particular sequence may be “heterologous” with respect to a cell or organism into which it is inserted (i.e., does not naturally occur in that particular cell or organism).

The expression “operably linked” means that the elements of the chimeric gene are linked to one another in such a way that their function is coordinated and allows expression of the coding sequence, i.e., they are functionally linked. By way of example, a promoter is functionally linked to another nucleotide sequence when it is capable of ensuring transcription and ultimately expression of the other nucleotide sequence. Two proteins encoding nucleotide sequences, e.g., a transit peptide encoding nucleic acid sequence and a nucleic acid sequence encoding a protein according to the disclosure, are functionally or operably linked to each other if they are connected in such a way that a fusion protein of a first and second protein or polypeptide can be formed.

A gene, e.g., a chimeric gene comprising a specific EIN5 allele of the disclosure, is said to be expressed when it leads to the formation of an expression product. An expression product denotes an intermediate or end product arising from the transcription and optionally translation of the nucleic acid, DNA or RNA, coding for such product. During the transcription process, a DNA sequence under control of regulatory regions, particularly the promoter, is transcribed into an RNA molecule.

As the skilled person will be well aware, various promoters may be used to promote the transcription of the nucleic acids encoding the functional EIN5 alleles of the disclosure. Such promoters include, for example, constitutive promoters, inducible promoters (e.g., stress-inducible promoters, drought-inducible promoters, hormone-inducible promoters, chemical-inducible promoters, etc.), tissue-specific promoters, developmentally regulated promoters and the like.

The chimeric gene of the disclosure may also comprise a 3′ end region, i.e., a transcription termination or polyadenylation sequence, operable in plant cells. As a transcription termination or polyadenylation sequence, use may be made of any corresponding sequence of bacterial origin, such as, for example, the nos terminator of Agrobacterium tumefaciens, of viral origin, such as, for example, the CaMV 35S terminator, or of plant origin, such as, for example, a histone terminator as described in published Patent Application EP 0 633 317 A1. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

### Methods for Producing Plants Having Functional, Mutant EIN5 Alleles in Plants

In specific embodiments, the introduction of the functional mutant EIN5 allele of the disclosure occurs in a plant having wild-type EIN5 alleles.

In a specific embodiment, the functional mutant EIN5 alleles of the disclosure can be introduced into the plant, e.g., through mutagenesis. “Mutagenesis,” as used herein, refers to the process in which plant cells are subjected to a technique that induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), T-DNA insertion mutagenesis (Azpiroz-Leehan et al. (1997) Trends Genet. 13:152-156), transposon mutagenesis (McKenzie et al. (2002) Theor. Appl. Genet. 105:23-33), or tissue culture mutagenesis (induction of somaclonal variations), or a combination of two or more of these. Thus, the desired mutagenesis of one or more EIN5 genes or EIN5 alleles may be accomplished by one of the above methods. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, plants are regenerated from the treated cells using known techniques. For instance, the resulting seeds may be planted in accordance with conventional growing procedures and following pollination seed is formed on the plants. Additional seed that is formed as a result of such pollination in the present or a subsequent generation may be harvested and screened for the presence of mutant-functional EIN5 alleles of the disclosure. Several techniques are known to screen for specific mutant alleles, e.g., DELETE-A-GENE™ (Delete-a-gene; Li et al., 2001, Plant J. 27:235-242) uses polymerase chain reaction (PCR) assays to screen for deletion mutants generated by fast neutron mutagenesis, TILLING (targeted induced local lesions in genomes; McCallum et al., 2000, Nat. Biotechnol. 18:455-457) identifies EMS-induced point mutations, etc.

In yet another embodiment, the mutant-functional EIN5 alleles can also be introduced via gene targeting techniques. The term “gene targeting” refers herein to directed gene modification that uses mechanisms such as double-stranded DNA break repair via non-homologous end-joining, homologous recombination, mismatch repair or site-directed mutagenesis. The method can be used to replace, insert and delete endogenous sequences or sequences previously introduced in plant cells. Methods for gene targeting can be found in, for example, WO 2006/105946 or WO 2009/002150. Double-stranded DNA breaks can be induced in a targeted manner using custom designed sequence-specific nucleases and nuclease systems such as meganucleases/homing endonucleases, zinc finger nucleases, TALENs or CRISPR/CAS (for a review, see Gaj et al., 2013, Trends Biotechnol. 31:397-405). In addition to making mutant alleles, such techniques can also be used to delete entire genes encoding proteins having the activity of a protein having any of the above amino acid sequences.

A mutant-functional EIN5 allele of the disclosure can also be introduced through introgression of a generated mutant-functional EIN5 allele into the plant.

### Isolation of Orthologous Plant Sequences of the Identified EIN5 Alleles

Based on the identified functional EIN5 allelic sequences of the disclosure, the skilled person can isolate homologous sequences or functional variants of the sequences disclosed herein using methods well known in the art, e.g., alignments, either manually or by using computer programs such as BLAST (Altschul et al. (1990), Journal of Molecular Biology 215:403-410), which stands for Basic Local Alignment Search Tool or ClustalW (Thompson et al. (1994), Nucleic Acid Res. 22:4673-4680) or any other suitable program that is suitable to generate sequence alignments. Homologous sequences as described above can comprise orthologous or paralogous sequences. Several different methods are known by those of skill in the art for identifying and defining these functionally homologous sequences. Three general methods for defining orthologous and paralogous genes are described; an ortholog, paralog or homolog may be identified by one or more of the methods described below. Orthologs and paralogs are evolutionarily related genes that have similar sequence and similar functions. Orthologs are structurally related genes in different species that are derived by a speciation event. Paralogs are structurally related genes within a single species that are derived by a duplication event. Within a single plant species, gene duplication may result in two copies of a particular gene, giving rise to two or more genes with similar sequence and often similar function known as paralogs. A paralog is, therefore, a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22:4673-4680; Higgins et al. (1996) Methods Enzymol. 266:383-402). Groups of similar genes can also be identified with pairwise BLAST analysis (Feng and Doolittle (1987) J. Mol. Evol. 25:351-360). Orthologous sequences can also be identified by a reciprocal BLAST strategy. Once an orthologous sequence has been identified, the function of the ortholog can be deduced from the identified function of the reference sequence. Orthologous genes from different organisms have highly conserved functions, and very often essentially identical functions (Lee et al. (2002) Genome Res. 12:493-502; Remm et al. (2001) J. Mol. Biol. 314:1041-1052). Paralogous genes, which have diverged through gene duplication, may retain similar functions of the encoded proteins. In such cases, paralogs can be used interchangeably with respect to certain embodiments of this disclosure (for example, transgenic expression of a coding sequence).

Functional variants or homologues of the sequences disclosed herein may also be identified and isolated by hybridization under stringent conditions using, as probes, identified nucleotide sequences or fragments thereof. For example, homologous sequences from other monocot species than the specific sequences disclosed herein are said to be substantially identical or essentially similar if they can be detected by hybridization under stringent, preferably highly stringent, conditions. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of, e.g., 100 nt) are, for example, those that include at least one wash in 0.2×SSC at 63° C. for 20 minutes, or equivalent conditions.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 minutes) at the hybridization temperature in 0.2-0.1×SSC, 0.1% SDS. “Moderate stringency conditions” refers to conditions equivalent to hybridization in the above-described solution but at about 60° C.-62° C. Moderate stringency washing may be done at the hybridization temperature in 1×SSC, 0.1% SDS. “Low stringency” refers to conditions equivalent to hybridization in the above-described solution at about 50° C.-52° C. Low stringency washing may be done at the hybridization temperature in 2×SSC, 0.1% SDS.

Example 8 depicts plant orthologs and EIN5 alleles.

In a specific embodiment, the chimeric genes comprising a functional, mutated EIN5-allele of the disclosure are incorporated in recombinant vectors. Typically, such recombinant vectors are plant transformation vectors further comprising a selectable marker or screenable marker gene.

“Selectable or screenable marker,” “selectable or screenable marker gene” or “reporter gene” includes any gene that confers a phenotype on a cell in which it is expressed to facilitate the identification and/or selection of cells that are transfected or transformed with a nucleic acid construct of the disclosure. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptll that phosphorylates neomycin and kanamycin, or hpt, phosphorylating hygromycin, or genes conferring resistance to, for example, bleomycin, streptomycin, tetracycline, chloramphenicol, ampicillin, gentamycin, geneticin (G418), spectinomycin or blasticidin), to herbicides (for example, bar that provides resistance to BASTA®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilization of xylose, or anti-nutritive markers such as the resistance to 2-deoxyglucose). Expression of visual marker genes results in the formation of color (for example, β-glucuronidase, GUS or β-galactosidase with its colored substrates, for example, X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can, for example, be used in mutants in which these genes are not functional by, for example, deletion by conventional methods. Furthermore, nucleic acid molecules encoding a selectable marker can be introduced into a host cell on the same vector that comprises the sequence encoding the polypeptides of the disclosure or used in the methods of the disclosure, or else in a separate vector. Cells that have been stably transfected with the introduced nucleic acid can be identified, for example, by selection (for example, cells that have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the disclosure for introducing the nucleic acids advantageously employs techniques that enable the removal or excision of these marker genes. One such method is what is known as co-transformation. The co-transformation method employs two vectors simultaneously for the transformation, one vector bearing the nucleic acid according to the disclosure and a second bearing the marker gene(s). A large proportion of transformants receives or, in the case of plants, comprises (up to 40% or more of the transformants) both vectors. In case of transformation with Agrobacteria, the transformants usually receive only a part of the vector, i.e., the sequence flanked by the T-DNA, which usually represents the expression cassette. The marker genes can be subsequently removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approximately 10%), the transposon jumps out of the genome of the host cell once transformation has successfully taken place and is lost. In a further number of cases, the transposon jumps to a different location. In these cases, the marker gene must be eliminated by performing crosses. In microbiology, techniques were developed that make possible, or facilitate, the detection of such events. A further advantageous method relies on what is known as recombination systems, whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has successfully taken place by expression of the recombinase. Further recombination systems are the HIN/HIX, FLP/FRT and REP/STB system (Tribble et al., J. Biol. Chem., 2000: 275:22255-22267; Velmurugan et al., J. Cell Biol., 2000: 149:553-566). A site-specific integration into the plant genome of the nucleic acid sequences according to the disclosure is possible. Similarly, marker genes can be excised using one or more rare-cleaving double-strand break-inducing enzymes such as meganucleases (naturally occurring or engineered to recognize a specific DNA sequence), zinc finger nucleases, TALE nucleases and the like, if recognition sites for such enzymes are present in the vicinity of the marker gene. Excision can occur via homologous recombination if homology regions flank the marker gene, or via non-homologous end-joining with two recognition sites flanking the marker gene.

For the purposes of the disclosure, “transgenic,” “transgene,” or “recombinant” means with regard to, for example, a nucleic acid sequence, an expression cassette, gene construct or a vector comprising the nucleic acid sequence or an organism transformed with the nucleic acid sequences, expression cassettes or vectors according to the disclosure. The term “nucleic acid molecule” as used interchangeably with the term “polynucleotide” in accordance with this disclosure, includes DNA, such as cDNA or genomic DNA, and RNA.

A “transgenic plant,” for the purposes of the disclosure, is thus understood as meaning, as above, that the nucleic acids used in the method of the disclosure (e.g., the chimeric genes) are not present in, or originating from, the genome of the plant, or are present in the genome of the plant but not at their natural locus in the genome of the plant, it being possible for the nucleic acids to be expressed homologously or heterologously. However, as mentioned, “transgenic” also means that, while the nucleic acids according to the disclosure or used in the inventive method are at their natural position in the genome of a plant, the sequence has been modified with regard to the natural sequence, and/or that the regulatory sequences of the natural sequences have been modified. “Transgenic” is preferably understood as meaning the expression of the nucleic acids according to the disclosure at an unnatural locus in the genome, i.e., where homologous or heterologous expression of the nucleic acids takes place. Preferred transgenic plants are mentioned herein.

The term “expression” or “gene expression” means the transcription of a specific gene or specific genes or specific genetic construct. The term “expression” or “gene expression,” in particular, means the transcription of a gene or genes or genetic construct into structural RNA (rRNA, tRNA) or mRNA with or without subsequent translation of the latter into a protein. The process includes transcription of DNA and processing of the resulting mRNA product.

The term “introduction” or “transformation,” as referred to herein, encompass the transfer of an exogenous polynucleotide into a host cell, irrespective of the method used for transfer. Plant tissue capable of subsequent clonal propagation, whether by organogenesis or embryogenesis, may be transformed with a genetic construct of this disclosure and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, mega-gametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (F. A. Krens et al. (1982) Nature 296:72-74; I. Negrutiu et al. (1987) Plant Mol. Biol. 8:363-373); electroporation of protoplasts (R. D. Shillito et al. (1985) Bio/Technol. 3:1099-1102); microinjection into plant material (A. Crossway et al. (1986) Mol. Gen. Genet. 202:179-185); DNA or RNA-coated particle bombardment (T. M. Klein et al. (1987) Nature 327:70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the disclosure to allow a suspension of transformed agrobacteria to act on the intact plant or at least on the flower primordia. The plant is subsequently grown on until the seeds of the treated plant are obtained (Clough and Bent, Plant J. (1998) 16:735-743). Methods for Agrobacterium-mediated transformation of rice include well-known methods for rice transformation, such as those described in any of the following: European patent application EP1198985; Aldemita and Hodges (Planta 199:612-617, 1996); Chan et al. (Plant Mol. Biol. 22(3):491-506, 1993); Hiei et al. (Plant J. 6(2):271-282, 1994), which disclosures are incorporated by reference herein as if fully set forth. In the case of corn transformation, the preferred method is as described in either Ishida et al. (Nat. Biotech. 14(6):745-50, 1996) or Frame et al. (Plant Physiol. 129(1):13-22, 2002), which disclosures are incorporated by reference herein as if fully set forth. The methods are further described by way of example in B. Jenes et al., “Techniques for Gene Transfer” in: Transgenic Plants, Vol. 1, Engineering and Utilization, eds. S. D. Kung and R. Wu, Academic Press (1993) 128-143; and in Potrykus, Annu. Rev. Plant Physiol. Plant Mol. Biol. (1991) 42:205-225). The nucleic acids or the construct to be expressed is preferably cloned into a vector, which is suitable for transforming Agrobacterium tumefaciens, for example, pBin19 (Bevan et al. (1984) Nucl. Acids Res. 12-8711). Agrobacteria transformed by such a vector can then be used in a known manner for the transformation of plants, such as plants used as a model, like Arabidopsis or crop plants such as, by way of example, tobacco plants, for example, by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16:9877, or is known inter alia from F. F. White, “Vectors for Gene Transfer in Higher Plants,” in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and, in particular, those cells that develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic (K. A. Feldman and M. D. Marks (1987), Mol. Gen. Genet. 208:1-9; K. Feldmann (1992) in C. Koncz, N.-H. Chua and J. Shell, eds, Methods in Arabidopsis Research, Word Scientific, Singapore, pp. 274-289). Alternative methods are based on the repeated removal of the inflorescences and incubation of the excision site in the center of the rosette with transformed agrobacteria, whereby transformed seeds can likewise be obtained at a later point in time (Chang (1994) Plant 1 5:551-558; Katavic (1994) Mol. Gen. Genet. 245:363-370). However, an especially effective method is the vacuum infiltration method with its modifications such as the “floral dip” method. In the case of vacuum infiltration of Arabidopsis, intact plants under reduced pressure are treated with an agrobacterial suspension (N. Bechthold (1993) C.R. Acad. Sci. Paris Life Science 316:1194-1199), while in the case of the “floral dip” method, the developing floral tissue is incubated briefly with a surfactant-treated agrobacterial suspension (S. J. Clough and A. F. Bent (1998) The Plant J. 16:735-743). A certain proportion of transgenic seeds are harvested in both cases, and these seeds can be distinguished from non-transgenic seeds by growing under the above-described selective conditions. In addition, the stable transformation of plastids is advantageous because plastids are inherited maternally in most crops, reducing or eliminating the risk of transgene flow through pollen. The transformation of the chloroplast genome is generally achieved by a process that has been schematically displayed in Klaus et al., 2004 (Nature Biotechnology 22(2):225-229). Briefly, the sequences to be transformed are cloned together with a selectable marker gene between flanking sequences homologous to the chloroplast genome. These homologous flanking sequences direct site-specific integration into the plastome. Plastidal transformation has been described for many different plant species and an overview is given in Bock (2001), “Transgenic plastids in basic research and plant biotechnology,” J. Mol. Biol. 2001 Sep. 21; 312 (3):425-38; or P. Maliga (2003), “Progress towards commercialization of plastid transformation technology,” Trends Biotechnol. 21:20-28. Further, biotechnological progress has recently been reported in the form of marker-free plastid transformants, which can be produced by a transient co-integrated maker gene (Klaus et al., 2004, Nature Biotechnology 22(2):225-229).

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally, after transformation, plant cells or cell groupings are selected for the presence of one or more markers, which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance, using Southern analysis, for the presence of the gene of interest, copy number and/or genomic organization. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells, clonal transformants (e.g., all cells transformed to contain the expression cassette), or grafts of transformed and untransformed tissues (e.g., in plants, a transformed rootstock grafted to an untransformed scion).

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

In some embodiments, the plant cell according to the disclosure is non-propagating or cannot be regenerated into a plant.

Plants that are particularly useful in the methods of the disclosure include, in particular, monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g., Brassica napus, Brassica rapa ssp. (canola, oilseed rape, turnip rape)), Cadaba farinosa, Camellia sinensis, Canna indica, Cannabis sativa, Capsicum spp., Carex data, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., Glycine max, Soja hispida or Soja max), Gossypium hirsutum, Helianthus spp. (e.g., Helianthus annuus), Hemerocallis fulva, Hibiscus spp., Hordeum spp. (e.g., Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g., Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

The choice of suitable control plants is a routine part of an experimental setup and may include corresponding wild-type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant,” as used herein, refers not only to whole plants, but also to plant parts, including seeds and seed parts.

The term “expression cassette” refers to any recombinant expression system for the purpose of expressing a nucleic acid sequence of the disclosure, in vitro or in vivo, constitutively or inducibly, in any cell, including, in addition to plant cells, prokaryotic, yeast, fungal, insect or mammalian cells. The term includes linear and circular expression systems. The term includes all vectors. The cassettes can remain episomal or integrate into the host cell genome. The expression cassettes can have the ability to self-replicate or not (i.e., drive only transient expression in a cell). The term includes recombinant expression cassettes that contain only the minimum elements needed for transcription of the recombinant nucleic acid.

The following non-limiting Examples describe methods and means according to the disclosure. Unless stated otherwise in the Examples, all techniques are carried out according to protocols standard in the art. The following examples are included to illustrate embodiments of the disclosure. Those of skill in the art should, in light of the present disclosure, appreciate that many changes can be made in the specific embodiments that are disclosed and still obtain a like or similar result without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### EXAMPLES

### Materials and Methods

1. Screen for Mutants Suppressing ERF6-Induced Dwarfism

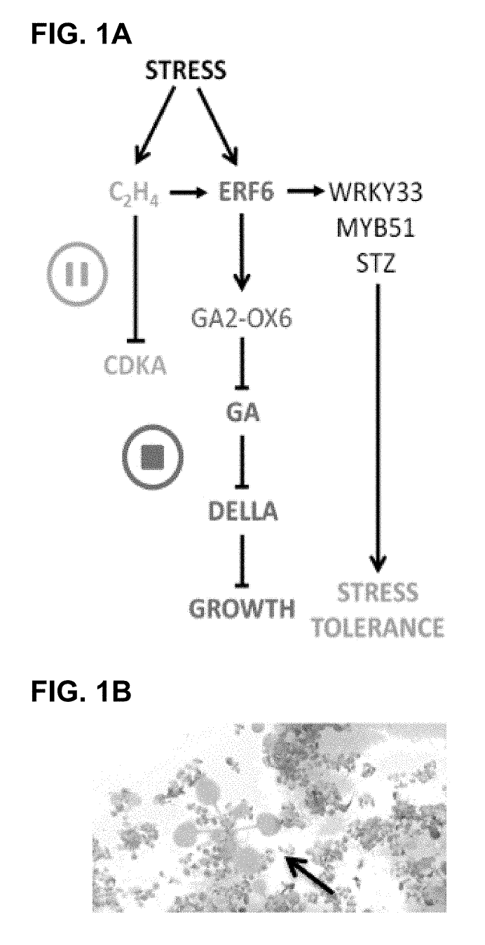

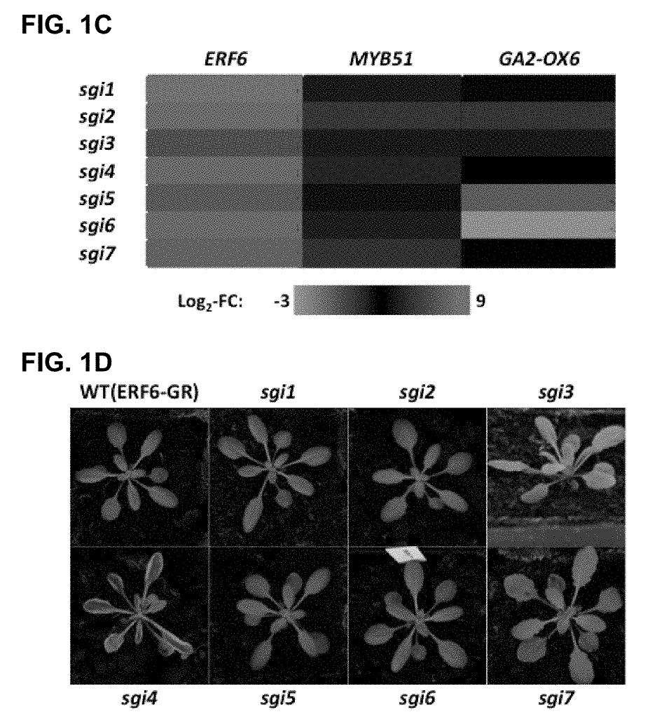

It has been shown in the art that leaf growth under stress is regulated by the ERF6-centered pathway in a GA/DELLA-dependent way (Dubois et al., 2013; Claeys et al., 2012) (FIG. 1A). Consistently, plants highly overexpressing the transcription factor ERF6 are extremely dwarfed, and show dark green, down-curling leaves. This easily detectable phenotype was used as a starting point for a forward genetics screen and subjected 10000 seeds of an ERF6-GR line (M1) to treatment with the mutagen 1-Methylsulfonyloxyethane (EMS). This commonly used mutagen introduces random and stable point mutations in the genome (Weigel and Glazebrook, 2006). Mutagenized seeds were selfed to obtain M2 mutants homozygous for both the ERF6-GR construct and for the introduced mutations, enabling the identification of recessive mutations. To screen for mutants suppressing the ERF6-induced phenotype, 20,000 Arabidopsis (M2) seeds were grown in vitro on medium containing dexamethasone (DEX) to induce the ERF6-overexpression, and on Kanamycin (Km). As the ERF6-GR construct contains a Kanamycin resistance marker, this step enabled removal of wild-type seeds that possibly contaminated the seed stock. In the presence of the inducing compound (DEX), the non-mutated plants and those that were mutated in a gene unrelated to growth were extremely dwarfed (FIG. 1B). In contrast, 12 plants able to repress the ERF6-phenotype because they were mutated in a gene related to ERF6-mediated growth regulation, grew normally. They were selected and selfed to obtain large amounts of non-segregating M3 seeds. The mutants were further crossed with each other to enable identification of allelic groups. In total, the 12 mutants were classified in seven different allelic groups: six groups each containing only one mutant, and one group containing six allelic mutants. For this last group of six mutants, a representative mutant was chosen for further analysis. The seven non-allelic mutants were named sgi1-sgi7 (Suppressor of ERF6-mediated Growth Inhibition).

To validate the mutants, they were first checked by PCR and sequencing for the presence of an intact ERF6-GR construct. Two mutants, named sgi1 and sgi2, were found to be mutated in the ERF6-domain of the ERF6-GR construct. This provoked missense mutations, respectively A270T and R285Q, which are situated in the DNA-binding AP2-domain of the ERF6 protein. However, because upon crossing with a wild-type, the progeny of both mutants segregated according to the 2^{nd} Mendelian law for two independent alleles, it was concluded that the mutation in ERF6 itself is not responsible for the suppression of the ERF6-induced dwarfism.

Secondly, investigation was performed as to whether the suppression of the ERF6-induced dwarfism is a specific suppression of the growth-regulatory pathway controlled by ERF6, or a more general abolishment of all functions of ERF6. As reported previously, ERF6 plays a dual role under stress with, next to the activation of the growth-inhibitory pathway, the induction of several stress tolerance-related transcription factors such as MYB51, STZ and WRKY33 (Dubois et al., 2013; Sakamoto et al., 2000, 2004; Gigolashvili et al., 2007; Jiang and Deyholos, 2009; Birkenbihl et al., 2012; Li et al., 2012; Niu et al., 2012; Dubois et al., 2013). Thus, expression analysis was performed by qRT-PCR to measure the capacity of ERF6 to induce the stress tolerance-related targets, and to confirm the inability to activate the growth-related target gene GA2-OX6. To induce the overexpression of ERF6, 15-day-old seedlings were exposed to dexamethasone-containing medium and RNA was extracted from the actively expanding third true leaf. It was observed that in 5 of the 7 mutants (sgi1-sgi4 and sgi7) the induction of GA2-OX6 by ERF6 no longer occurs, while the stress tolerance genes were still induced (FIG. 1C). Amongst these five mutants are the two mutants sgi1 and sgi2 with mutated ERF6, confirming that the functionality of ERF6 was not affected by the in cis mutation. The other two mutants, sgi2 and sgi6, were also affected in the induction of the tolerance-related genes, but showed a remarkable down-regulation of the growth-inhibitory GA2-OX6 gene and were, for that reason, kept in the screen (FIG. 1C).

Interestingly, when growing the seven different mutants in soil, under non-DEX conditions, it was observed that several mutants show additional phenotypes, on top of the capacity to suppress the ERF6-GR phenotype (FIG. 1D). For example, the sgi3 mutant exhibits a clear shade avoidance phenotype accompanied by early flowering. The sgi4 mutant has a very striking epinastic phenotype and shows trichomes at both sides of the rosette leaves, while these are generally only observed on the adaxial leaf side. This mutant also shows defective formation of siliques, which often remain very small and contain only a few seeds. Importantly, this sgi7 mutant showed visible increase in rosette size. Larger rosettes were sometimes also observed for sgi1 and sgi6, although this was not reproducible. Thus, it was concluded that seven non-allelic mutants are able to suppress ERF6-induced dwarfism through different molecular mechanisms and that some of these mutants show interesting rosette phenotypes on top of the capacity to suppress the ERF6-phenotype.
2. Identification of the Causal Genes

To identify the gene mutated in each of the seven mutants, each mutant (in Col-0 background) was outcrossed with the Arabidopsis Ler-1 accession. About 3000 F2 seeds were screened in vitro on growth medium containing both Km and DEX. Upon segregation, 400 mutants showing a normal growth phenotype, and thus containing at least a single copy of the ERF6-GR and being homozygous for the causal mutation, were selected and pooled for bulk segregant analysis using Shoremap (Schneeberger et al., 2009). The following genes were found to carry the causal mutation in the respective mutants (see Table 1).

The mutants sgi1 and sgi2 were mutated in, respectively, AT1G54820 and AT2G36350. Both genes encode putative kinases without apparent homology, but both have previously been associated with the response in Arabidopsis leaves upon infection by the Gemini virus (Ascencio-Ibanez et al., 2008). Because ERF6 C-terminally possesses a phosphorylation site necessary for proper ERF6 function (Nakano et al., 2006; Meng et al., 2013; Wang et al., 2013), the fact that the two mutants mutated in the ERF6-domain of the ERF6-GR both show a causal mutation in a kinase enzyme raises the question whether an impaired post-translational modification of ERF6 by these mutant kinases and/or by the mutant ERF6 could explain the suppression of the ERF6-induced dwarfism. This is rather unlikely as the mutations are not situated in the putative phosphorylation site, but the exact molecular mechanism and putative connection between ERF6 and these two kinases certainly deserves further consideration.

The mutation in sgi3 still remains to be identified. The clear shade avoidance phenotype and the early flowering could suggest a mutation in a gene involved in GA-biosynthesis or signaling such as, for example, overexpression of the GA20-OX1 GA-biosynthetic enzyme shows a comparable phenotype. The same hold for mutant sgi5, in which the causal mutation still remains unidentified.

The mutant sgi4 appeared to be mutated in AT3G05040, a gene encoding the HASTY protein. A G≧A mutation in this gene generates a premature stop codon and probably a truncated protein. The HASTY protein is a nuclear membrane-located protein from the importin/exportin family that imports/exports different substrates such as mRNA or small proteins to or out of the nucleus (Telfer and Poethig, 1998; Bollman et al., 2003). Importantly, the HASTY gene has already been identified in multiple EMS screens, including some based on GR-lines (Allen et al., 2013; REFs). As the functionality of GR lines is relying on proper transport of the constitutively overexpressed fusion protein to the nucleus, it can be speculated that HASTY is involved in this translocation and that truncated HASTY proteins fail in this process. However, it then remains elusive how induction of the stress-related ERF6 target genes still occurs in sgi4. It might be speculated that few ERF6-GR proteins sporadically reach the nucleus through other transporters, which can be sufficient for the activation of some but not all ERF6 targets. Another hypothesis might involve putative protein-interaction partners of ERF6, which could be necessary to activate a subset of the ERF6-targets (such as the GA2-OX6) and of which the transport to the nucleus is HASTY-dependent. This is, however, completely speculative. Mutants in the HASTY gene have previously been reported to exhibit the same phenotype as observed for sgi4, including the highly pronounced epinasty and the trichomes on the lower side of the leaf, indicating a faster maturation of the shoot (Bollman et al., 2003).

In the mutant sgi6, the mutated gene AT3G27670 encodes the transmembrane receptor-like protein RESURRECTION1 (RST1). This gene has previously been reported to negatively affect the cuticular wax biosynthesis, as mutants in RST1 have increased levels of cuticular wax with an altered fatty acid composition (Chen et al., 2005). As mutants produce 70% of shrunken and unviable seeds (which was also observed for the sgi6 mutant), the RST1 gene is also thought to play a role in embryo development (Chen et al., 2005). Finally, the RST1 protein is a positive regulator of plant defense against biotrophic pathogens, and a negative regulator of defense against necrotrophes (Mang et al., 2009). In sgi6, the mutation in the splice acceptor site of the first intron generates a postponed splicing engendering a frameshift and thus likely results in loss-of-function of RST1.

Finally, the sgi7 mutant carried a mutation in the gene encoding EIN5, also named XRN4. EIN5 is a 5′≧3′ exoribonuclease and degrades transcripts carrying preferential EIN5 binding sites (Souret et al., 2004; Rymarquis et al., 2011). The EIN5 enzyme has been involved in the ethylene signaling pathways as reverse genetic mutants of EIN5 are ethylene insensitive, and no longer show the typical triple response of ethylene-treated seedlings grown in the dark (Van der Straeten et al., 1993; Roman et al., 1995; Olmedo et al., 2006; Potuschak et al., 2006). Consistently with this identification, the sgi7 mutant did not show triple response upon germination in the dark on medium containing the ethylene precursor 1-aminocyclopropane-1-carboxylic acid (ACC). The sgi7 mutant looked particularly interesting as it showed a visible increase in leaf size and as it was the representative mutant of the group of six allelic mutants, further called sgi7.1-sgi7.6. Therefore, characterization of these mutants in more detail was chosen.

TABLE 1
Identification of the mutations in the seven non-allelic
EMS mutants.

| | | | Predicted effect | Additional |
|---|---|---|---|---|
| Mutant | Mutated gene | Gene name | on protein | phenotype |
| sgi1 | AT1G54820 + | Putative kinase | | / |
| | mutation in ERF6 | | | |
| sgi2 | AT2G36350 + | Putative kinase | | / |
| | mutation in ERF6 | | | |
| sgi3 | | | | Shade |
| | | | | avoidance |
| sgi4 | AT3G05040 | HASTY | Truncated | Epinasty |
| | | | protein | |
| sgi5 | | | | / |
| sgi6 | AT3G27670 | RST1 | Frameshift | / |
| sgi7 | AT1G54490 | EIN5 | Multiple, | Larger, |
| | | | See Table 2 | serrated |
| | | | | rosette |
| | | | | leaves |

3. Multiple Mutations Identified in Conserved Domains of EIN5

As the promising sgi7 mutant (from now on named “sgi7.1”) was the representative of an allelic group of six mutants, the exact mutation in each of the five other mutants was also identified (see Table 2). The mutations were mapped on six different sites on the coding sequence of EIN5 and had predicted different effects on the protein sequence; in sgi7.1, sgi7.2 and sgi7.4, the point mutation could result in an amino acid substitution, while in sgi7.3 and sgi7.5, the mutation is likely to generate a nonsense mutation. Finally, in sgi7.6, the mutation was situated in the splice acceptor site of the 14^{th} intron, which could generate incorrect splicing and finally result in a frameshift.

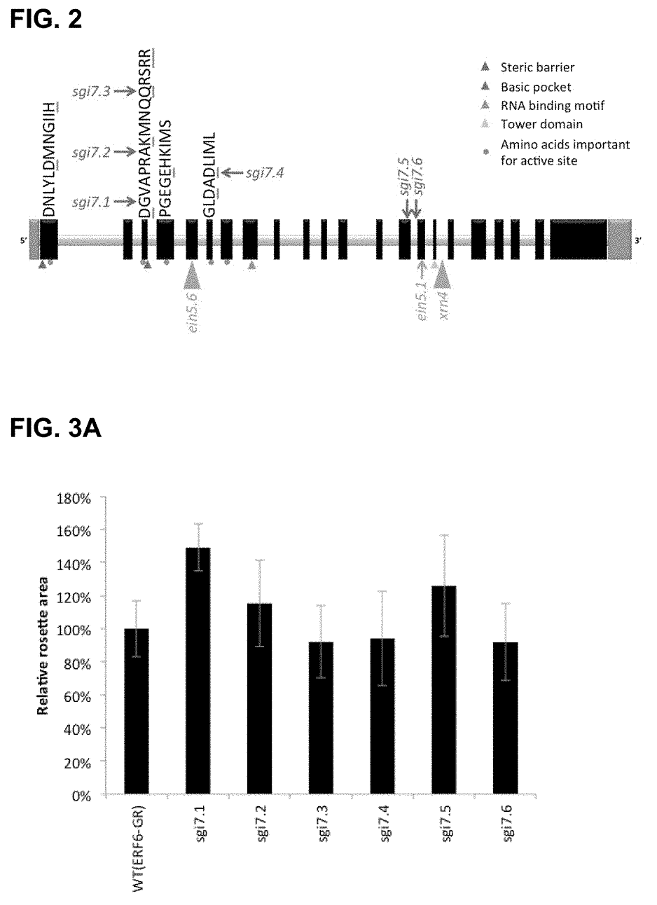

The EIN5/XRN4 protein is a rather huge protein of 947 amino acids with relatively well-conserved orthologs in Drosophila and yeast (Nagarajan et al., 2013). The protein structure of the yeast XRN1 has recently been described, and the N-terminal half of the protein, containing the most important functional domains, is thought to be conserved between the orthologs. At its N-terminal, the exoribonuclease EIN5 contains several small well-conserved domains, such as a steric barrier to prevent penetrance of dsRNA, a basic pocket for substrate stabilization, and an RNA-binding motif (FIG. 2). Next to this, the N-terminal side contains several extremely well-conserved amino acids that are thought to be crucial for proper functionality of the active site and that are situated in short stretches of very well-conserved amino acids. Interestingly, several of the identified mutations are localized within these very conserved and, thus, potentially important motifs (FIG. 2).

TABLE 2
Position of the mutations in the EMS mutants for
EIN5 and their effect.

| Mutant | Nt position | Mutation Nt | AA position | Mutation AA |
|---|---|---|---|---|
| sgi7.1 | 314 | G ≧ A | 105 | G ≧ E |
| sgi7.2 | 329 | C ≧ T | 110 | A ≧ V |
| sgi7.3 | 343 | C ≧ T | 115 | Q ≧ STOP |
| sgi7.4 | 706 | G ≧ A | 236 | D ≧ N |
| sgi7.5 | 1476 | C ≧ T | 526 | Q ≧ STOP |

| | | | |
|---|---|---|---|
| sgi7.6 | 1603 | G ≧ A | Splice acceptor site mutated |

Six mutations were identified on different positions of the EIN5 coding sequence. Based on the validated mutations, the effect on protein level was predicted: three of them generate a missense mutation, two a nonsense mutation and one generates an incorrect splicing variant, probably resulting in a frameshift. Nt=nucleotide, AA=amino acid.
4. Different Alleles with Different Effects on Leaf Growth

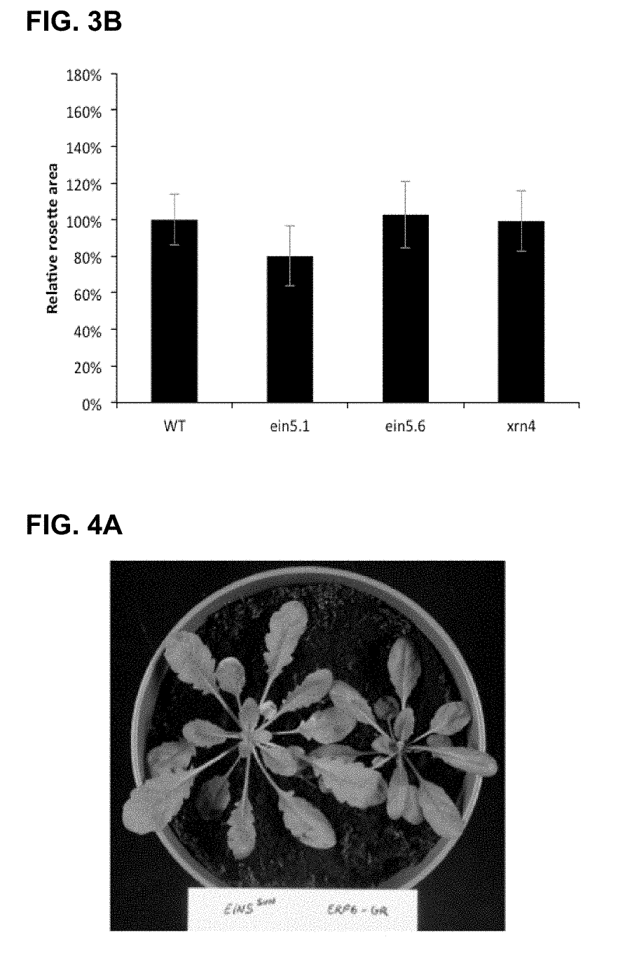

All identified mutants are able to suppress ERF6-induced dwarfism but interestingly, some of them show additional phenotypes under non-dex conditions (FIG. 1D). To investigate whether all identified mutations in EIN5 had a similar or different effect on Arabidopsis leaf growth, all mutants were simultaneously grown in soil for 22 days and rosette size was measured by making leaf series. Interestingly, the six sgi7 alleles showed different levels of increase in leaf size (FIG. 3A). The most pronounced increase in leaf size was observed for the sgi7.1 mutant, with an average increase of 49% (p=2E-9). The mutants sgi7.5 and sgi7.2 also showed a significant increase in final rosette area of, respectively, 26% and 15%. In contrast, the mutants sgi7.3 and sgi7.6 were not larger than the WT (ERF6-GR). To further evaluate these growth phenotypes, three independent previously used mutants of EIN5 (ein5.1, ein5.6 and xrn4) were phenotyped in a similar way. These three mutants are confirmed loss-of-function mutants as they carry a 1-bp deletion causing a frameshift, a T-DNA insertion in the fifth exon, and a T-DNA insertion in an intron, respectively (FIG. 2) (Olmedo et al., 2006). Although these mutants have been extensively studied, their leaf growth has never been characterized in detail. At 22 days after stratification (DAS), the final rosette area was unaltered as compared to wild-type rosette size (FIG. 3B). The observation that in these loss-of-function mutants the leaf size is not increased reinforces our hypothesis that the mutations in sgi7.1, sgi7.2 and sgi7.5 generate EIN5 alleles likely to alter the activity of this protein.
5. The EIN5^{G105E} Allele of sgi7.1 Enhances Leaf Growth

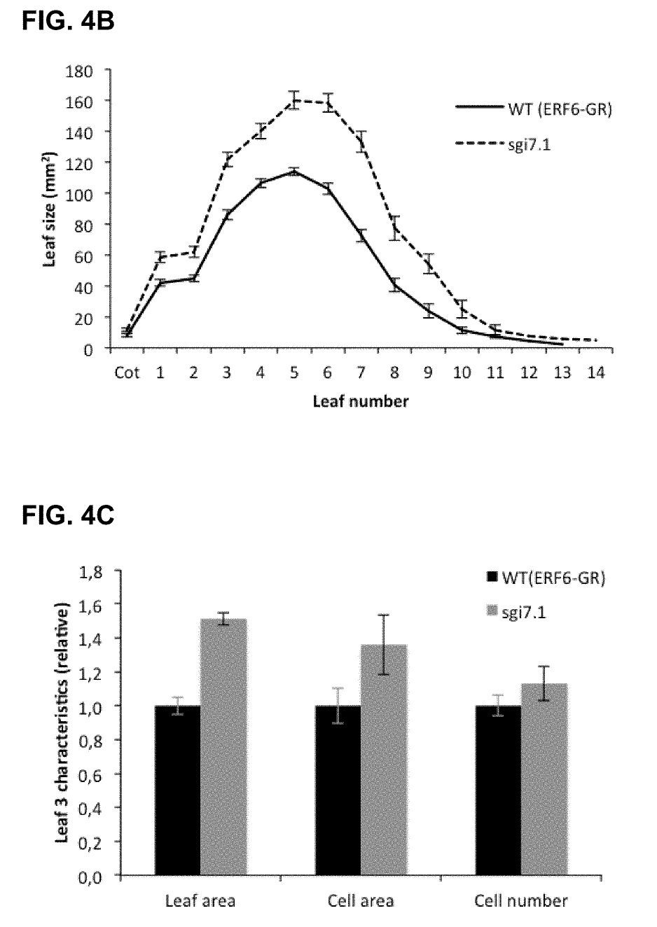

To unravel the mechanisms behind the stimulation of leaf growth in the sgi7 mutants, further focus was put on the mutant with the largest rosette area, sgi7.1. Detailed analysis showed that this mutant is on average 53% larger resulting from an increased size of all leaves (FIGS. 4A and 4B). To uncover the cellular mechanism behind it, cellular drawings of the abaxial epidermal layer were made to determine whether increased leaf growth results from more and/or larger cells. A pronounced increase in cell area was observed, with an average increase of 36% (FIG. 4C). The increase in cell area was, however, insufficient to explain the increased leaf size (51% for the third leaf), but the cell number was only slightly and not significantly increased in all biological repeats. Thus, it was concluded that the leaf size increase in the sgi7.1 mutant mainly results from an increase in cell area and that the EIN5^{G105E} allele mainly affects the expansion phase of leaf development, although small effects on cell division cannot entirely be excluded.
6. The sgi7.1 Mutant is More Tolerant to Mild Drought Stress

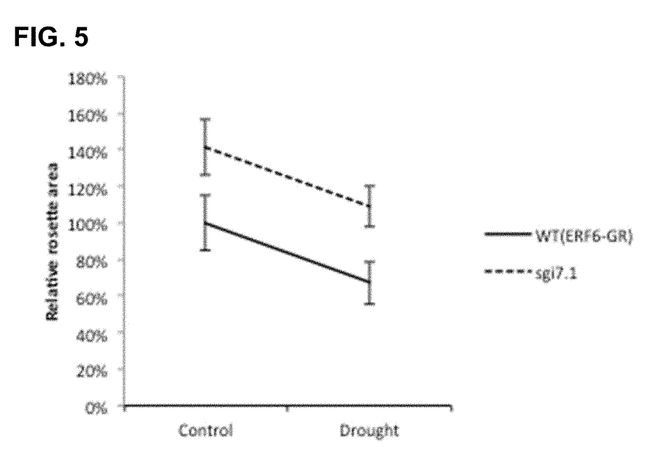

Because the growth-inhibitory pathway on which the forward genetics screen is based is mainly active under adverse environmental conditions reducing rosette growth, the sgi7.1 mutant was exposed to mild drought stress. Mutant and control were grown on the Weighing Imaging and Watering Machine (WIWAM) under a well-watered regime for 11 days and then exposed to a mild drought stress regime reducing wild-type rosette size by about 30-40% (Skirycz et al., 2011). Rosette size was measured at 22 DAS. In three independent biological repeats, the drought-induced growth inhibition was less pronounced in the sgi7.1 mutant (−23% in sgi7.1 compared to −33% in WT) (FIG. 5). As this mutant already shows a growth advantage under normal conditions, this resulted in sgi7.1 mutants being under mild drought equally large as wild-type under control conditions. Thus, it was concluded that the EIN5^{G105E} allele not only positively affects growth under control conditions, but also under mild drought stress.
7. Introduction of an EIN5 Mutant-Functional Allele in Zea mays

The maize homolog of EIN5 is GRMZM2G099630 and the nucleotide sequence is depicted in SEQ ID NO:14 (see Example 8). In order to introduce mutations in the DNA coding for the conserved sequence (GDVAP) (SEQ ID NO:17), the CRISPR technology was used by stably transforming plants with a construct containing UBI::CAS9 and U6::gRNA, with gRNA containing the sequence targeting the conserved box (gatggtgttgctccaaggg) (SEQ ID NO:18). The gRNA will guide the CAS9 to the target sequence, which will be cleaved. NHEJ will repair the nick while causing insertions, deletions, and substitutions. The resulting lines will be genotyped and the lines with no frame shift will be further analyzed and phenotyped.
8. Plant Orthologues of EIN5 Genes

SEQ ID NO:1: wild-type EIN5 protein sequence of Arabidopsis thaliana

Conserved sequences between different plant orthologs of EIN5 are underlined. The positions of the sgi7.1-sgi7.5 allelic variants in the sequence are marked as “[].”

MGVPAFYRWLADRYPKSISDVVEEEPTDGGRGDLIPVDITRPNPNGFEFD
NLYLDMNGIIHPCFHPEGKPAPATYDDVFKSMFEYIDHLFTLVRPRKILY
LAIDGVAPRAKMNQQRSRRFRAAKDAAEAEAEEERLRKDFEMEGQILSAK
EKAETCDSNVITPGTPFMAILSVALQYYIQSRLNHNPGWRYVKVILSDSN
VPGEGEHKIMSYIRLQRNLPGFDPNTRHCLYGLDADLIMLSLATHEVHFS
ILREVITYPGQQEKCFVCGQTGHFASDCPGKSGSNNAAADIPIHKKKYQF
LNIWVLREYLQYELAIPDPPFMINFERIIDDFVFLCFFVGNDFLPHMPTL
EIREGAINLLMHVYRKEFTAMGGYLTDSGEVLLDRVEHFIQAVAVNEDKI
FQKRTRIKQSMDNNEEMKQRSRRDPSEVPPEPIDDKIKLGEPGYKERYYA
EKFSTTNPEETEQIKQDMVLKYVEGLCWVCRYYYQGVCSWQWFYPYHYAP
FASDLKNLPDLEITFFIGEPFKPFDQLMGTLPAASSNALPGEYRKLMTDP
SSPILKFYPADFELDMNGKRFAWQGIAKLPFIEEKLLLAATRKLEETLTV
EEQQRNSVMLDLLYVHPAHPLGQRILQYYHFYQHMPPHECLPWMIDPNSS
QGMNGFLWFSERNGFQTRVDSPVNGLPCIEQNRALNVTYLCPAKHSHISE
PPRGAIIPDKILTSVDIKPFPPLWHEDNSNRRRQARDRPQVVGAIAGPSL
GEAAHRLIKNTLNMKSSTGAASGLIDPNGYYRNVPGNYSYGGVNRPRAPG
PSPYRKAYDDDSSYYYGKYNNSTQGTFNNGPRYPYPSNGSQDYNRNYNSK
IVAEQHNRGGLGAGMSGLSIEDNGRSKQLYSSYTEAANANLNPLPSPPTQ
WIGTQPGGNFVGGYYRDGVGYSETNGKSVKKVIYQAKTQPSHRGANL

The position of the identified functional mutant alleles of EIN5 in A. thaliana is shown in Table 3 below.

TABLE 3
position of mutated amino acids in SEQ ID NO: 1 for
the different functional allelic mutants of EIN5.

| Mutant | Nt position | Mutation Nt | AA position | Mutation AA |
|---|---|---|---|---|
| sgi7.1 | 314 | G ≧ A | 105 | G ≧ E |
| sgi7.2 | 329 | C ≧ T | 110 | A ≧ V |
| sgi7.3 | 343 | C ≧ T | 115 | Q ≧ STOP |
| sgi7.4 | 706 | G ≧ A | 236 | D ≧ N |
| sgi7.5 | 1476 | C ≧ T | 526 | Q ≧ STOP |

| | | | |
|---|---|---|---|
| sgi7.6 | 1603 | G ≧ A | Splice acceptor site mutated |

SEQ ID NO:2 (nucleotide sequence of the Arabidopsis thaliana wild-type EIN5 gene)

SEQ ID NO:3 (Glycine max) amino acid sequence of EIN5 protein

SEQ ID NO:4 (Glycine max) nucleotide sequence of EIN5 gene

SEQ ID NO:5 (Malus domesticus) amino acid sequence of EIN5 protein

SEQ ID NO:6 (Malus domesticus) nucleotide sequence of EIN5 gene

SEQ ID NO:7 (Oryza sativa japonica) amino acid sequence of EIN5 protein

SEQ ID NO:8 (Oryza sativa japonica) nucleotide sequence of the EIN5 gene

SEQ ID NO:9 (Oryza sativa indica) amino acid sequence of EIN5 protein

SEQ ID NO:10 (Oryza sativa indica) nucleotide sequence of EIN5 gene

SEQ ID NO:11 (Populus trichocarpa) protein sequence of EIN5

SEQ ID NO:12 (Populus trichocarpa) nucleotide sequence of EIN5 gene

SEQ ID NO:13 amino acid sequence of Zea mays EIN5 protein

SEQ ID NO:14 nucleotide sequence of Zea mays EIN5 gene

### REFERENCES

Achard, P., F. Gong, S. Cheminant, M. Alioua, P. Hedden and P. Genschik (2008). “The cold-inducible CBF1 factor-dependent signaling pathway modulates the accumulation of the growth-repressing DELLA proteins via its effect on gibberellin metabolism.” Plant Cell 20(8):2117-2129.

Allen, R. S., K. Nakasugi, R. L. Doran, A. A. Millar and P. M. Waterhouse (2013). “Facile mutant identification via a single parental backcross method and application of whole genome sequencing based mapping pipelines.” Frontiers in Plant Science 4.

Anastasiou, E. and M. Lenhard (2007). “Growing up to one's standard.” Current Opinion in Plant Biology 10(1): 63 -69.

Andriankaja, M., S. Dhondt, S. De Bodt, H. Vanhaeren, F. Coppens, L. De Milde, P. Muhlenbock, A. Skirycz, N. Gonzalez, G. T. S. Beemster and D. Inze (2012). “Exit from Proliferation during Leaf Development in Arabidopsis thaliana: A Not-So-Gradual Process.” Developmental Cell 22(1):64-78.

Ascencio-Ibanez, J. T., R. Sozzani, T. J. Lee, T. M. Chu, R. D. Wolfinger, R. Cella and L. Hanley-Bowdoin (2008). “Global analysis of Arabidopsis gene expression uncovers a complex array of changes impacting pathogen response and cell cycle during geminivirus infection.” Plant Physiology 148(1):436-454.

Baerenfaller, K., C. Massonnet, S. Walsh, S. Baginsky, P. Buhlmann, L. Hennig, M. Hirsch-Hoffmann, K. A. Howell, S. Kahlau, A. Radziejwoski, D. Russenberger, D. Rutishauser, I. Small, D. Stekhoven, R. Sulpice, J. Svozil, N. Wuyts, M. Stitt, P. Hilson, C. Granier and W. Gruissem (2012). “Systems-based analysis of Arabidopsis leaf growth reveals adaptation to water deficit.” Molecular Systems Biology 8.

Birkenbihl, R. P., C. Diezel and I. E. Somssich (2012). “Arabidopsis WRKY33 Is a Key Transcriptional Regulator of Hormonal and Metabolic Responses toward Botrytis cinerea Infection.” Plant Physiology 159(1):266-285.

Bollman, K. M., M. J. Aukerman, M. Y. Park, C. Hunter, T. Z. Berardini and R. S. Poethig (2003). “HASTY, the Arabidopsis ortholog of exportin 5/MSNS, regulates phase change and morphogenesis.” Development 130(8):1493-1504.

Chen, X. B., S. M. Goodwin, X. L. Liu, X. L. Chen, R. A. Bressan and M. A. Jenks (2005). “Mutation of the RESURRECTION1 locus of Arabidopsis reveals an association of cuticular wax with embryo development.” Plant Physiology 139(2):909-919.

Claeys, H. and D. Inze (2013). “The Agony of Choice: How Plants Balance Growth and Survival under Water-Limiting Conditions.” Plant Physiology 162(4):1768-1779.

Claeys, H., A. Skirycz, K. Maleux and D. Inze (2012). “DELLA Signaling Mediates Stress-Induced Cell Differentiation in Arabidopsis Leaves through Modulation of Anaphase-Promoting Complex/Cyclosome Activity.” Plant Physiology 159(2):739-+.

Dinneny, J. R. (2008). “Cell identity mediates the response of Arabidopsis roots to abiotic stress (vol. 320, pg. 942, 2008).” Science 322(5898):44-44.

Donnelly, P. M., D. Bonetta, H. Tsukaya, R. E. Dengler and N. G. Dengler (1999). “Cell cycling and cell enlargement in developing leaves of Arabidopsis.” Developmental Biology 215(2):407-419.

Dubois, M., A. Skirycz, H. Claeys, K. Maleux, S. Dhondt, S. De Bodt, R. Vanden Bossche, L. De Milde, T. Yoshizumi, M. Matsui and D. Inze (2013). “ETHYLENE RESPONSE FACTOR6 Acts as a Central Regulator of Leaf Growth under Water-Limiting Conditions in Arabidopsis.” Plant Physiology 162(1):319-332.

Gigolashvili, T., B. Berger, H. P. Mock, C. Muller, B. Weisshaar and U. I. Fluegge (2007). “The transcription factor HIG1/MYB51 regulates indolic glucosinolate biosynthesis in Arabidopsis thaliana.” Plant Journal 50(5):886-901.

Gonzalez, N., H. Vanhaeren and D. Inze (2012). “Leaf size control: complex coordination of cell division and expansion.” Trends in Plant Science 17(6):332-340.

Harb, A., A. Krishnan, M. M. R. Ambavaram and A. Pereira (2010). “Molecular and Physiological Analysis of Drought Stress in Arabidopsis Reveals Early Responses Leading to Acclimation in Plant Growth.” Plant Physiology 154(3):1254-1271.

Jiang, Y. Q. and M. K. Deyholos (2009). “Functional characterization of Arabidopsis NaCl-inducible WRKY25 and WRKY33 transcription factors in abiotic stresses.” Plant Molecular Biology 69(1-2):91-105.

Li, G. J., X. Z. Meng, R. G. Wang, G. H. Mao, L. Han, Y. D. Liu and S. Q. Zhang (2012). “Dual-Level Regulation of ACC Synthase Activity by MPK3/MPK6 Cascade and Its Downstream WRKY Transcription Factor during Ethylene Induction in Arabidopsis.” Plos Genetics 8(6).

Mang, H. G., K. A. Laluk, E. P. Parsons, D. K. Kosma, B. R. Cooper, H. C. Park, S. AbuQamar, C. Boccongelli, S. Miyazaki, F. Consiglio, G. Chilosi, H. J. Bohnert, R. A. Bressan, T. Mengiste and M. A. Jenks (2009). “The Arabidopsis RESURRECTION1 Gene Regulates a Novel Antagonistic Interaction in Plant Defense to Biotrophs and Necrotrophs.” Plant Physiology 151(1):290-305.

Meng, X. Z., J. Xu, Y. X. He, K. Y. Yang, B. Mordorski, Y. D. Liu and S. Q. Zhang (2013). “Phosphorylation of an ERF Transcription Factor by Arabidopsis MPK3/MPK6 Regulates Plant Defense Gene Induction and Fungal Resistance.” Plant Cell 25(3):1126-1142.

Nagaraj an, V. K., C. I. Jones, S. F. Newbury and P. J. Green (2013). “XRN 5′≧3′ exoribonucleases: Structure, mechanisms and functions.” Biochimica Et Biophysica Acta-Gene Regulatory Mechanisms 1829(6-7) : 590-603.

Nakano, T., K. Suzuki, T. Fujimura and H. Shinshi (2006). “Genome-wide analysis of the ERF gene family in Arabidopsis and rice.” Plant Physiology 140(2):411-432.

Niu, C. F., W. Wei, Q. Y. Zhou, A. G. Tian, Y. J. Hao, W. K. Zhang, B. A. Ma, Q. Lin, Z. B. Zhang, J. S. Zhang and S. Y. Chen (2012). “Wheat WRKY genes TaWRKY2 and TaWRKY19 regulate abiotic stress tolerance in transgenic Arabidopsis plants.” Plant Cell and Environment 35(6):1156-1170.

Olmedo, G., H. W. Guo, B. D. Gregory, S. D. Nourizadeh, L. Aguilar-Henonin, H. J. Li, F. Y. An, P. Guzman and J. R. Ecker (2006). “ETHYLENE-INSENSITIVE5 encodes a 5′≧3′ exoribonuclease required for regulation of the EIN3-targeting F-box proteins EBF1/2.” Proc. Natl. Acad. Sci. U.S.A. 103(36): 13286-13293.

Potuschak, T., A. Vansiri, B. M. Binder, E. Lechner, R. D. Vierstra and P. Genschik (2006). “The exoribonuclease XRN4 is a component of the ethylene response pathway in Arabidopsis.” Plant Cell 18(11):3047-3057.

Roman, G., B. Lubarsky, J. J. Kieber, M. Rothenberg and J. R. Ecker (1995). “Genetic-Analysis of Ethylene Signal-Transduction in Arabidopsis-Thaliana—5 Novel Mutant Loci Integrated into a Stress-Response Pathway.” Genetics 139(3):1393-1409.

Rymarquis, L. A., F. F. Souret and P. J. Green (2011). “Evidence that XRN4, an Arabidopsis homolog of exoribonuclease XRN1, preferentially impacts transcripts with certain sequences or in particular functional categories.” RNA-a Publication of the RNA Society 17(3):501-511.

Sakamoto, H., T. Araki, T. Meshi and M. Iwabuchi (2000). “Expression of a subset of the Arabidopsis Cys(2)/His(2)-type zinc-finger protein gene family under water stress.” Gene 248(1-2):23-32.

Sakamoto, H., K. Maruyama, Y. Sakuma, T. Meshi, M. Iwabuchi, K. Shinozaki and K. Yamaguchi-Shinozaki (2004). “Arabidopsis Cys2/His2-type zinc-finger proteins function as transcription repressors under drought, cold, and high-salinity stress conditions.” Plant Physiology 136(1):2734-2746.

Schachtman, D. P. and J. Q. D. Goodger (2008). “Chemical root to shoot signaling under drought.” Trends in Plant Science 13(6):281-287.

Schneeberger, K., S. Ossowski, C. Lanz, T. Juul, A. H. Petersen, K. L. Nielsen, J. E. Jorgensen, D. Weigel and S. U. Andersen (2009). “SHOREmap: simultaneous mapping and mutation identification by deep sequencing.” Nature Methods 6(8):550-551.

Seki, M., A. Matsui, J. M. Kim, J. Ishida, M. Nakajima, T. Morosawa, M. Kawashima, M. Satou, T. K. To, Y. Kurihara, E. Kaminuma, T. Endo, Y. Mochizuki, N. Kobayashi, T. Toyoda and K. Shinozaki (2007). “Arabidopsis whole-genome transcriptome analysis under drought, cold, high-salinity, and ABA treatment conditions using tiling array and 454 sequencing technology.” Plant and Cell Physiology 48: S8-S8.

Skirycz, A., H. Claeys, S. De Bodt, A. Oikawa, S. Shinoda, M. Andriankaja, K. Maleux, N. B. Eloy, F. Coppens, S. D. Yoo, K. Saito and D. Inze (2011). “Pause-and-Stop: The Effects of Osmotic Stress on Cell Proliferation during Early Leaf Development in Arabidopsis and a Role for Ethylene Signaling in Cell Cycle Arrest.” Plant Cell 23(5):1876-1888.

Skirycz, A., S. De Bodt, T. Obata, I. De Clercq, H. Claeys, R. De Rycke, M. Andriankaja, O. Van Aken, F. Van Breusegem, A. R. Fernie and D. Inze (2010). “Developmental Stage Specificity and the Role of Mitochondrial Metabolism in the Response of Arabidopsis Leaves to Prolonged Mild Osmotic Stress.” Plant Physiology 152(1):226-244.

Skirycz, A. and D. Inze (2010). “More from less: plant growth under limited water.” Current Opinion in Biotechnology 21(2): 197-203.

Skirycz, A., K. Vandenbroucke, P. Clauw, K. Maleux, B. De Meyer, S. Dhondt, A. Pucci, N. Gonzalez, F. Hoeberichts, V. B. Tognetti, M. Galbiati, C. Tonelli, F. Van Breusegem, M. Vuylsteke and D. Inze (2011). “Survival and growth of Arabidopsis plants given limited water are not equal.” Nature Biotechnology 29(3):212-214.

Souret, F. F., J. P. Kastenmayer and P. J. Green (2004). “AtXRN4 degrades mRNA in Arabidopsis and its substrates include selected miRNA targets.” Molecular Cell 15(2): 173-183.

Telfer, A. and R. S. Poethig (1998). “HASTY: a gene that regulates the timing of shoot maturation in Arabidopsis thaliana.” Development 125(10):1889-1898.

Umezawa, T., M. Fujita, Y. Fujita, K. Yamaguchi-Shinozaki and K. Shinozaki (2006). “Engineering drought tolerance in plants: discovering and tailoring genes to unlock the future.” Current Opinion in Biotechnology 17(2): 113 -122.

Vanderstraeten, D., A. Djudzman, W. Vancaeneghem, J. Smalle and M. Vanmontagu (1993). “Genetic and Physiological Analysis of a New Locus in Arabidopsis That Confers Resistance to 1-Aminocyclopropane-1-Carboxylic Acid and Ethylene and Specifically Affects the Ethylene Signal-Transduction Pathway.” Plant Physiology 102(2):401-408.

Verslues, P. E., M. Agarwal, S. Katiyar-Agarwal, J. Zhu and J. K. Zhu (2006). “Methods and concepts in quantifying resistance to drought, salt and freezing, abiotic stresses that affect plant water status. (vol. 45, pg. 523, 2006).” Plant Journal 46(6):1092-1092.

Vlieghe, K., V. Boudolf, G. T. S. Beemster, S. Maes, Z. Magyar, A. Atanassova, J. D. Engler, R. De Groodt, D. Inze and L. De Veylder (2005). “The DP-E2F-like gene DEL1 controls the endocycle in Arabidopsis thaliana.” Current Biology 15(1):59-63.

Wang, P. C., Y. Y. Du, X. L. Zhao, Y. C. Miao and C. P. Song (2013). “The MPK6-ERF6-ROS-Responsive cis-Acting Element7/GCC Box Complex Modulates Oxidative Gene Transcription and the Oxidative Response in Arabidopsis.” Plant Physiology 161(3):1392-1408.

Weigel, D., J. Glazebrook (2006). Adapted from “Obtaining Mutants,” Chapter 2 in Arabidopsis by Detlef Weigel and Jane Glazebrook. Cold Spring Harbor Laboratory Press, Cold Spring Harbor, N.Y., USA, 2002.

Xiong, L. M., K. S. Schumaker and J. K. Zhu (2002). “Cell signaling during cold, drought, and salt stress.” Plant Cell 14:S165-S183.

1. A functional allele of EIN5 having its splice acceptor site mutated at nucleotide position 1603 in SEQ ID NO:2 or in a corresponding plant orthologous sequence thereof, or a functional allele encoding a mutant protein selected from the group consisting of:
a protein having at least one amino acid change in the sequence DGVAPRAKMNQQRSRR present in SEQ ID NO:1 or in a corresponding plant orthologous sequence thereof,
a protein having at least one amino acid sequence change in the sequence GLDADLIML present in SEQ ID NO:1 or in a corresponding plant orthologous sequence thereof, and
a protein having an amino acid change at position 526 in SEQ ID NO:1 or in a corresponding plant orthologous sequence thereof.

2. The functional allele of claim 1, wherein the functional allele has a splice acceptor site mutated at nucleotide position 1603 in SEQ ID NO:2 from G to A, or in a corresponding position of a plant orthologous sequence thereof, or wherein the functional allele encodes a functional protein selected from the group consisting of:
a protein having an amino acid change of G to E in position 105 of SEQ ID NO:1, or in a corresponding position in a plant orthologous sequence thereof,
a protein having an amino acid change of A to V in position 110 of SEQ ID NO:1, or in a corresponding position in a plant orthologous sequence thereof,
a protein having an amino acid change of Q to STOP in position 115 of SEQ ID NO:1, or in a corresponding position in a plant orthologous sequence thereof,
a protein having an amino acid change of D to N in position 236 of SEQ NO:1, or in a corresponding position in a plant orthologous sequence thereof, and
a protein having an amino acid change of Q to a STOP-codon in position 526 of SEQ ID NO: NO:1, or in a corresponding position in a plant orthologous sequence thereof.

3. A chimeric gene comprising:
a plant expressible promoter,
the mutant, functional EIN5 allele according to claim 1, and
a functional terminator sequence.

4. A mutant plant, or a cell, part, seed or progeny thereof, having knock-out EIN5 genes, the plant, cell, part, seed or progeny comprising:
the chimeric gene according to claim 3.

5. A mutant plant, or a cell, part, seed or progeny thereof, having its wild-type EIN5 genes replaced by mutant, functional EIN5 alleles in a homozygous state wherein said alleles include the functional allele of claim 1.

6. The plant of claim 4, wherein the plant's yield is increased by at least 20% compared to a plant having wild-type EIN5 alleles.

7. The plant of claim 4, wherein the drought tolerance is increased compared to a plant having wild-type EIN5 alleles.

8. The plant of claim 5, wherein the plant's yield is increased by at least 20% compared to a plant having wild-type EIN5 alleles.

9. The plant of claim 5, wherein the plant's drought tolerance is increased compared to a plant having wild-type EIN5 alleles.

10. A chimeric gene comprising:
a plant expressible promoter,
the mutant, functional EIN5 allele of claim 2, and
a functional terminator sequence.

11. A mutant plant, plant cell, plant part, plant seed, or progeny thereof, having knock-out EIN5 genes, the plant, cell, part, seed or progeny comprising:
the chimeric gene of claim 10.

12. A mutant plant, or a cell, part, seed or progeny thereof, having its wild-type EIN5 genes replaced by mutant, functional EIN5 alleles in a homozygous state wherein the alleles include the functional allele of claim 2.

13. The functional allele of claim 1, wherein the functional allele has its splice acceptor site mutated at nucleotide position 1603 in SEQ ID NO:2.

14. The functional allele of claim 1, wherein the functional allele encodes a protein having at least one amino acid change in sequence DGVAPRAKMNQQRSRR present in SEQ ID NO:1.

15. The functional allele of claim 1, wherein the functional allele encodes a protein having at least one amino acid sequence change in sequence GLDADLIML present in SEQ ID NO:1.

16. The functional allele of claim 1, wherein the functional allele encodes a protein having an amino acid change at position 526 in SEQ ID NO:1.

17. The functional allele of claim 2, wherein the functional allele has a splice acceptor site mutated at nucleotide position 1603 in SEQ ID NO:2 from G to A.

18. The functional allele of claim 2, wherein the functional allele encodes a protein having an amino acid change of G to E in position 105 of SEQ ID NO:1.

19. The functional allele of claim 2, wherein the functional allele encodes a protein having an amino acid change of A to V in position 110 of SEQ ID NO:1.

20. The functional allele of claim 2, wherein the functional allele encodes a protein having an amino acid change of Q to STOP in position 115 of SEQ ID NO:1.

21. The functional allele of claim 2, wherein the functional allele encodes a protein having an amino acid change of D to N in position 236 of SEQ ID NO:1.

22. The functional allele of claim 2, wherein the functional allele encodes a protein having an amino acid change of Q to a STOP codon in position 526 of SEQ ID NO: 1.
